(19) United States
(12) Patent Application Publication
(10) Pub. No.: US 2024/0076630 A1
(43) Pub. Date: Mar. 7, 2024

(54) METHODS FOR GENERATING NEURAL TISSUE AND USES THEREOF

(71) Applicant: President and Fellows of Harvard College, Cambridge, MA (US)

(72) Inventors: Giorgia Quadrato, Cambridge, MA (US); Paola Arlotta, Cambridge, MA (US)

(21) Appl. No.: 18/231,190

(22) Filed: Aug. 7, 2023

Related U.S. Application Data
(63) Continuation of application No. 16/067,815, filed on Jul. 2, 2018, now abandoned.
(60) Provisional application No. 62/423,566, filed on Nov. 17, 2016.
(60) Provisional application No. 62/273,795, filed on Dec. 31, 2015.

(51) Int. Cl. C12N 5/071; C12N 5/079; C12N 5/0793

(52) U.S. Cl. CPC C12N 5/0697; C12N 5/0618; C12N 5/0619; C12N 5/0622; C12N 2501/115; C12N 2501/13; C12N 2503/04; C12N 2506/02; C12N 2506/45; C12N 2513/00; C12N 2533/90

(57) ABSTRACT

Disclosed herein are 3-D neural tissue structures or “brain organoids” created from human pluripotent cells (e.g., stem cells) differentiated into neuronal cell types that include cortical and subcortical neuronal subtypes along with sensory cells. Also disclosed herein are methods for the in vitro generation of 3-D neural tissue structures capable of sensory perception, methods for generating a “brain organoid-machine interface” (BOMI), and methods for screening of molecular, cellular and network-level defects associated with complex mental diseases through use of patient-derived induced pluripotent stem cells.

### RELATED APPLICATIONS

This application is a continuation application of U.S. application Ser. No. 16/067,815, filed Jul. 2, 2018, which is a national stage filing under 35 U.S.C. 371 of International Application No. PCT/US2016/069546, filed Dec. 30, 2016, which claims the benefit of U.S. Provisional Application No. 62/273,795, filed Dec. 31, 2015, and U.S. Provisional Application No. 62/423,566, filed Nov. 17, 2016. The entire teachings of the above applications are incorporated herein by reference. International Application No. PCT/US2016/069546 was published under PCT Article 21(2) in English.

### BACKGROUND OF THE INVENTION

Human brain development and neurodevelopmental disorders are poorly understood processes. Studies using human and primate brain tissue have been limited by practical and ethical concerns related to tissue availability, expansion and manipulation. In addition, rodent studies have been limited by inherent differences in the development, architecture and function of the rodent brain compared to a human brain.

Reductionist in vitro models of the developing human brain have emerged in recent years in the form of 3D human brain organoids and spheroids derived from pluripotent stem cells, which are amenable to large-scale production and genetic engineering. 1 These systems offer an opportunity to study both normal brain development and complex human diseases that affect multiple cell types, the interactions between them, and the function of neuronal circuits.

However, various issues arise, including the incomplete understanding of the cellular composition of brain organoids and the potential of organoids to generate the regional and cellular diversity present in the brain. Progress is further hindered by the fact that the diversity of cell types present in these models is likely extremely high, requiring an unprecedented number of single-cell gene expression profiles to ascertain the cellular heterogeneity of the system. Another critical issue is the ability to understand whether 3D brain organoids can continue to develop in culture past early developmental events, e.g., to enable not only the generation of endogenous cellular diversity, but also the maturation of neuronal networks.

### SUMMARY OF THE INVENTION

The present invention provides methods for the in vitro generation of a 3-D neural tissue structure that is capable of sensory perception and response. A 3-D neural tissue structure capable of sensory perception and response or “brain organoid” is created from human pluripotent cells (e.g., stem cells) differentiated into neuronal cell types that preferably include cortical and subcortical neuronal subtypes along with sensory cells. The brain organoid forms complex neural networks that connect neurons with sensory cells in physiologically relevant circuits. The complex neural networks have spontaneous activity and respond to physiological stimuli such as light.

The invention also provides methods for generating a “brain organoid-machine interface” (BOMI). A brain organoid can be interfaced with a computer, whereby spontaneous and induced sensory circuit activity input is analyzed. The software can output instructions to the stimulus-generating device in a loop, thereby mimicking sensory-response learning.

The invention also provides methods for screening of molecular, cellular and network-level defects associated with diseases, including complex mental diseases, through use of patient-derived induced pluripotent stem cells. Brain organoids generated from patient cells can be screened for defects in cellular composition, as well as for spontaneous network activity. Brain organoids can also be screened for response to stimulus and performance during BOMI learning sessions (“capacity to learn”). Such screening methods can also assess the impact of agents on brain function, particularly at the molecular, cellular and/or network level.

In some embodiments, the present inventions are directed to three dimensional neural tissue compositions, the compositions comprising a cerebral organoid exhibiting discrete brain regions comprising one or more sensory receptors and cells, wherein said one or more sensory receptors are capable of detecting a corresponding stimulus.

In some embodiments, the compositions further include other cell types including, but not limited to, one or more of microglia, oligodendrocytes, endothelial cells, cells of the immune system and stromal cells. In some embodiments, the compositions further include differentiated human cell types selected from the group consisting of cortical neurons, subcortical neurons, and sensory cells. In some embodiments, the sensory cells are one or more cells bearing one or more sensory receptors selected from the group consisting of photoreceptors, auditory receptors, olfactory receptors, tactile receptors, and taste receptors. In some embodiments, the one or more sensory receptors are capable of responding to a detected stimulus.

In some embodiments, the compositions further include a neural circuit. In some embodiments, the sensory cells form a neural network with one or more additional cells within the organoid. In some embodiments, the neural network includes a functional connection between the neural circuit and the sensory cell. In some embodiments, the neural network is capable of exhibiting a response to external physiological stimuli. For example, the physiological stimuli may be one or more stimuli selected from the group consisting of light, sound, taste, smell, temperature and touch.

In some embodiments, the present inventions are directed to in vitro methods of producing a three dimensional neural tissue organoid, the methods comprising forming embryoid bodies from cells, applying a medium comprising hESC medium and neural induction medium to the formed embryoid bodies, generating neuroectodermal tissue from the embryoid bodies, transferring the neuroectodermal tissue to a protein mixture and maintaining in a cerebral organoid differentiation medium for 3 to 5 days to form neural tissue, transferring the neural tissue to a tissue culture vessel and maintaining in the cerebral organoid differentiation medium for 28 to 32 days, and replacing the cerebral organoid differentiation medium with a cerebral organoid differentiation medium supplemented with neurotrophin BDNF and maintaining neural tissue in the supplemented organoid differentiation medium for a time sufficient to produce a three dimensional neural tissue organoid. In some embodiments, the methods of the claimed invention enable growth of neural tissue having longer axons and/or more mature neurons than previously known methods.

In some embodiments, the cells are human cells. In some embodiments, the cells are human stem cells. In some embodiments, the cells are human induced pluripotent stem cells. In some embodiments, the cells are patient-derived induced pluripotent stem cells. In some embodiments, the patient-derived induced pluripotent stem cells are derived from a patient exhibiting a complex disease affecting brain activity. In some embodiments, the complex disease is selected from the group consisting of epilepsy, schizophrenia, bipolar disorder and autism spectrum disorder (ASD).

In some embodiments, the tissue culture vessel is a bioreactor, a spinner flask, or an orbital shaker. In some embodiments, the protein mixture is MATRIGEL (gelatinous protein mixture secreted by Engelbreth-Holm-Swarm (EHS) mouse sarcoma cells). In some embodiments, the neural tissue maintained in the supplemented organoid differentiation medium continues to maturate for at least 10 months, or, in other embodiments, at least a year or more.

In some embodiments, the present inventions are directed to generating three dimensional neural tissue compositions by the methods disclosed herein and isolating one or more cell types from the composition. The isolated one or more cell types may be further cultured and optionally used for screening or disease modeling.

In some embodiments, the present inventions are directed to methods for screening for diseases, the methods comprising generating a three dimensional neural tissue composition comprising a cerebral organoid from patient-derived induced pluripotent stem cells, and screening for dysregulation of spontaneous activity or defects of stimulus-induced activity. In some embodiments, the cerebral organoids are co-cultured with other cell types including, but not limited to, one or more of microglia, oligodendrocytes, endothelial cells, cells of the immune system and stromal cells. In some embodiments, the diseases are neurological, neuropsychological, neuropsychiatric, neurodegenerative, or neuropsychopharmacological diseases. In some embodiments, the diseases are neuropsychiatric diseases.

In still other embodiments, disclosed herein are brain organoid-machine interfaces comprising a multi-probe electrode array configured to collect electrophysiological signals from neural tissue, a first processor connected to the multi-probe electrode array, a second processor coupled to a stimulus-generating device, and machine executable instructions configured to decode circuit response and instruct feedback stimulation to the sensory generating device.

In some embodiments, the first processor is configured to collect and store the electrophysiological signals. In some embodiments, the stimulus-generating device is a light-emitting diode (LED). In some embodiments, the stimulus-generating device generates a stimulus selected from the group consisting of a visual stimulus, an auditory stimulus, an olfactory stimulus, a taste stimulus, a temperature and a touch stimulus. In some embodiments, the brain organoid-machine interface measures the neural tissue for spontaneous activity in response to the stimulus. In certain embodiments, the brain organoid machine interface measures the neural tissue for network activity in response to the stimulus. In some embodiments, the brain organoid-machine interface screens the neural tissue for dysregulation of spontaneous activity in response to the stimulus. In some embodiments, the brain organoid-machine interface screens the neural tissue for defects of synaptic and network activity in response to the stimulus. In some embodiments, the brain organoid-machine interface screens the neural tissue for defects in performance of the neural tissue during a learning session.

Also disclosed herein are three dimensional neural tissue compositions comprising a cerebral organoid. In some embodiments, the three dimensional neural tissue compositions comprising a cerebral organoid are cultured for at least 9 months and exhibiting discrete brain regions comprising one or more sensory receptors and cells. In some aspects, the cerebral organoid is cultured for 9 to 13 months. In some embodiments, the cerebral organoid is cultured for less than 9 months. In some embodiments, the cerebral organoid is cultured for about 6, 7, 8, 9, 10, 11, 12, 13, 14, 15, 16 months or more.

Also disclosed herein are three dimensional neural tissue compositions comprising a mature cerebral organoid exhibiting dendritic spine-like structures. Further disclosed herein, are cerebral organoid compositions comprising spontaneously-active neurons and neuronal networks.

In some embodiments, the cerebral organoid compositions comprise mature retinal tissue. The cerebral organoid compositions may comprise one or more of each cell type of the human retina.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D show large-scale, single-cell sequencing demonstrating development of a broad spectrum of cell types in human brain organoids. FIG. 1A provides a schematic for long-term culture of brain organoids and age of organoids at time of analyses. Dissociated human iPSCs are seeded at day 0 into round-bottom well plates to allow EB formation (day 2-5). After two-step neural induction (day 6-10), EBs are embedded in Matrigel (day 10) and transferred to spinning bioreactors (day 15) for long-term culture. BDNF is added to the culture medium starting at 1 month. Immunohistochemistry (IHH), single cell RNA-sequencing (Drop-seq), electrophysiology (E-phys) and electron microscopy (EM) were used to analyze organoids at distinct time points. FIG. 1B provides a t-SNE plot of single cell mRNA sequencing data from organoids at 6 months in culture. A total of 66,889 cells were clustered into 10 distinct groups. FIG. 1C provides a t-SNE plot of the forebrain cluster (c4), subclustered into 30 distinct groups (continuously shaded from magenta to teal). FIG. 1D shows a t-SNE plot of the retina cluster (c5), subclustered into 34 distinct groups (continuously shaded from yellow to brown).

FIGS. 2A-2D demonstrate human brain organoids contain subclasses of forebrain and retina cells. FIG. 2A shows subclustering of the forebrain cluster (c4). Five major cell subtypes were identified: radial glia (7 subclusters), interneurons (1 subcluster), intermediate progenitors (1 subcluster), corticofugal neurons (2 subclusters), or callosal neurons (8 subclusters). FIG. 2B illustrates expression of genes identified as retina cell subtype markers across the retinal subclusters shown in FIG. 2C (see FIG. 6B for sources). FIG. 2C shows subclustering of the retina cluster (c5). Six broad cell subtypes were identified as either Mueller glia (3 subclusters), photoreceptors (4 subclusters), retinal ganglion cells (2 subclusters), bipolar cells (3 subclusters), amacrine cells (4 subclusters), and pigmented epithelium (2 subclusters). Color coding is same as FIG. 2B. FIG. 2D illustrates expression of rhodopsin (RHO) and the pan-neuronal marker MAP2 in 6 month organoids by immunohistochemistry. Scale bars, 250 μm (left panel), 20 μm (right panels).

FIGS. 3A-3K demonstrate extended culture permits development of mature cell types including differentiated photoreceptors. FIG. 3A shows reclustering of CRX⁺ cells using the combined 3 month and 6 month data sets identifies 12 clusters. FIG. 3B shows cluster representation of the two ages indicates that cluster c5 is present only in 6 month organoids. FIG. 3C illustrates genes related to rod phototransduction are highly differentially expressed in the photoreceptor cluster (c5; marked by brown box) among CRX⁺ cells. FIG. 3D provides a heatmap of differentially-expressed genes with the maturation-related DAVID annotations “neuronal projections”, “long-term potentiation”, and “neuronal differentiation” in corticofugal projection neurons from 3 vs 6 month old organoids. FIGS. 3E-3K demonstrate synapse and dendritic spine development in organoids. FIG. 3E shows expression of the synaptic marker SYN1 is absent at 1 month and appears with organoid maturation by 3 months. FIG. 3F shows immunohistochemical detection of the synaptic proteins VGAT and VGLUT1 in a 6 month old organoid. FIG. 3G shows EM image of one slice from an 8 month old organoid (outer surface at the top). The red frame highlights the reconstructed area used in FIGS. 3H-3K. Scale bar, 10 μm. FIG. 3H provides example EM images of synaptic structures in the reconstructed volume. Scale bars, 1 μm. FIG. 3I provides 3D renderings of all traced axons (blue) and dendrites (red) that establish synapses in the serial EM volume. Scale bar, 1 μm. FIG. 3J shows dendrite with two spines (orange) making synapses with two axons (blue and green). Synaptic vesicles are shown in yellow. FIG. 3K shows synaptic contact sites (yellow) on the 29 spines identified in the volume. Scale bar, 1 μm.

FIGS. 4A-4H demonstrate human brain organoids develop spontaneously active neuronal networks that can be modulated by sensory stimulation of light-sensing cells. FIG. 4A provides a schematic of an experimental outline (see Methods). Extracellular recordings were performed on 4 and 8 month old organoids. Right image shows the recording chamber; inset shows close up image of the silicon probe (single arrowhead) and the organoid (double arrowhead). FIG. 4B illustrates mean spike rate for spontaneous activity (at least 15 min recording, n=31). FIG. 4C provides example inter-spike interval (top) and auto-correlogram (bottom) plots for spontaneous activity recorded from 4 prototypical units; 1 ms bins. FIG. 4D (top panel) illustrates spike-train cross-correlograms (0.5 ms bins). A mono-synaptic connection is identified by the presence of a positive peak, with a short time lag (<5 ms). Red line, estimated mean spike rate. Dotted blue line, statistical threshold for the identification of connected pairs (see Methods). FIG. 4D (bottom panel) illustrates mean spiking rate is attenuated by NBQX (20 μM, n=9 units; * p<0.05, two-tailed Wilcoxon signed rank test). FIG. 4E (left panels) show example population rate histograms (1 s bins), for organoids displaying non-stationary firing pattern (top left); and for organoids with a more homogenous population firing rate (bottom left). The raster plots for 3 example isolated units are shown above each histogram. FIG. 4E (right panels) shows plot of the fano factor calculated across the whole recording versus the fano factor calculated during the upstate (top right). The shaded region highlights the 99% confidence bounds for a whole recording fano factor of 1. Also shown is a plot of mean spike rate against the fano factor (n=31, from 6 eight month old organoids) (bottom right). Fano factors that are outside the expected 99% confidence bounds are plotted in red; those that are within the 99% confidence bounds are plotted in green. FIG. 4F illustrates during up-states, synchronized units are recruited with a reproducible temporal structure. Top panel shows mean firing rate of spike-trains aligned to burst onset; inset shows raster plots for three example bursts (spikes for 3 isolated units are color coded). Middle panel shows spike train auto-correlogram (color) and cross-correlogram (gray). Bottom panel shows mean spike waveforms recorded by silicon probes (peak response is plotted in color). Spatial arrangement of traces reflects probe geometry (top trace=most superficial). Scale bars, 2 ms, 50 μV FIG. 4G provides example spike rate histograms (1s bins) for two units, showing the effect of stimulation with 530 nm light (green bars): 200 ms steps of 530 nm light, delivered at 0.2 Hz (30 stimuli at 30 μW/cm², 30 stimuli at 100 μW/cm², and 30 stimuli at 300 μW/cm²). FIG. 4H shows light stimulation increases c-fos (red) expression in 8 month old organoids; rod-like cells are indicated by rhodopsin staining (green). Scale bars, 250 μm (low magnification), 20 μm (high magnification).

FIGS. 5A-5C illustrate time-course of expression of selective marker genes in human brain organoids. FIG. 5A shows one month old brain organoids exhibit early brain regionalization, expressing markers of forebrain, midbrain, and hindbrain progenitors. FIG. 5B shows expression of markers for progenitor, neuronal and glial populations over 1 to 9 months of culture. FIG. 5C demonstrates expression of progenitor and neuronal subtype-specific markers of dorsal forebrain shows progenitor cells lining ventricle-like structures at 1 month and the formation of layered structures resembling cortical laminae at 5 months, with segregated expression of upper-and deep-layer projection neuron markers. Scale bars, 250 (low magnification), 20 μm (high magnification).

FIGS. 6A-6B demonstrate decoding of the identity of cell types within the main clusters. FIG. 6A provides a heatmap showing expression of markers for selected cell types across the 10 main clusters in 6 month organoids. FIG. 6B provides a table of references used for cluster identification in FIG. 6A.

FIGS. 7A-7D illustrate ASD- and Schizophrenia-risk genes are expressed in brain organoids with cell type specificity. FIGS. 7A and 7B show t-SNE plots of the 6 month dataset showing representative examples of cells that express selected ASD-linked (FIG. 7A) or SCZ-linked (FIG. 7B) genes. A high number of genes are enriched in the forebrain cluster. FIG. 7C provides a heatmap of average difference across the forebrain subclusters of ASD-linked genes identified as differentially expressed compared to the entire dataset. FIG. 7D provides a heatmap of average difference across the forebrain subclusters of SCZ-linked genes identified as differentially expressed compared to the entire dataset.

FIGS. 8A-8K demonstrate electron microscopy of an 8 month old human brain organoid. FIGS. 8A-8C illustrate whole organoid before vibratome sectioning shows regional structures. Red line shows approximate location of the vibratome slice. FIG. 8D shows example 40 nm section. FIG. 8E shows regular finger-like membrane stack could indicate development of the outer segment of a retinal rod cell. FIG. 8F shows stacked endoplasmic reticulum. FIG. 8G shows example synapse with crystalline-looking arrangement of vesicles. FIG. 8H shows large cell body with muscle fibers highlighted in blue. Enlarged red square region shows muscle fibers with sarcomeres. Scale bar, 10 μm (low magnification) and 1 μm (enlargement). FIG. 8I shows several cell bodies with different morphologies and cytoplasm content highlighted in color. Scale bar, 10 μm. FIG. 8J shows one of the dark cell bodies showing as dark spots on the organoid surface. Scale bar, 10 μm. FIG. 8K shows cells reaching to the surface of the organoid. Scale bar, 10 μm.

### DETAILED DESCRIPTION OF THE INVENTION

The present invention provides methods for the in vitro generation of a 3-D neural tissue structure that is capable of sensory perception. In certain embodiments the neural tissue structure is also capable of response to sensory stimulation. In one embodiment, the brain organoid includes cortical and subcortical neurons, produced from the in vitro differentiation of cells (e.g., human cells such as human stem cells or other pluripotent, multipotent or totipotent cells). The generated organoid forms and comprises complex neural network circuits, which contain sensory cells. The network circuits include, but are not limited to, cortical neurons, subcortical neurons and functional sensory cells such as photoreceptors, olfactory receptors, and other functional sensory cells (e.g., auditory receptors, tactile receptors, etc). The network circuits may display spontaneous activity, as well as stimulus-induced activity.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant to include any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell type: internal organs, skin, bones, blood, and connective tissue are all made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell” by which is meant a cell that is present in or obtained from an organism other than an embryo or a fetus or results from proliferation of such a cell in vitro.

As used herein, the term “adult cell” refers to a cell found throughout the body after embryonic development.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “organoid” refers to a three-dimensional organ-bud grown in vitro and in isolation from an intact organism. Organoids may be derived from stem cells (e.g., embryonic stem cells, induced pluripotent stem cells, etc.). Various organoids may be formed including, but not limited to, cerebral organoids, thyroid organoids, intestinal organoids, testicular organoids, hepatic organoids, pancreatic organoids, gastric organoids, epithelial organoids, lung organoids, kidney organoids, retina organoids, inner ear organoids, and pituitary organoids.

The terms “cerebral organoid”, “3D brain tissue” or “brain organoid” are used interchangeably herein and refer to organoids that have anatomical features that resemble mammalian brains. The cerebral organoids may include synthesized tissues that contain several types of nerve cells. Cerebral organoids can be produced using human pluripotent stem cells (hPSCs). The general methodology for producing cerebral organoids includes culturing hPSCs under conditions suitable for the development of an embryoid body. The cell culture is then induced to form a neuroectoderm, and the neuroectoderm is grown in a protein matrix. The neuroectoderm begins to proliferate and grow, and is transferred to a tissue culture vessel where the cerebral organoids will continue to develop. Cerebral organoids may differentiate into one or more of various neural tissue types, such as the optic cup, hippocampus, ventral parts of the telencephalon and dorsal cortex.

The term “embryoid bodies” is synonymous with “aggregate bodies”. The terms refer to aggregates of differentiated and undifferentiated cells that appear when iPS cells overgrow in plated or suspension cultures.

The term “phenotype” refers to one or a number of total biological characteristics that define the cell or organism under a particular set of environmental conditions and factors, regardless of the actual genotype.

The term “pluripotent” as used herein refers to a cell with the capacity to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as an ectodermal stem cell), which in turn can differentiate into other types of precursor cells further down the pathway (such as a neural ectodermal cell), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties include unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting at least one embryoid body or a precursor thereof with a differentiation medium or agent) is intended to include incubating the differentiation medium and/or agent and the cell together in vitro (e.g., adding the differentiation medium or agent to cells in culture). In some embodiments, the term “contacting” is not intended to include the in vivo exposure of cells to the compounds as disclosed herein that may occur naturally in a subject (i.e., exposure that may occur as a result of a natural physiological process). The step of contacting at least one embryoid body or a precursor thereof with a differentiation medium or agent as in the embodiments related to the production of neural tissue can be conducted in any suitable manner. For example, the cells may be treated in adherent culture, or in suspension culture. In some embodiments, the cells are treated in conditions that promote cell clustering. The disclosure contemplates any conditions which promote cell clustering. Examples of conditions that promote cell clustering include, without limitation, suspension culture in low attachment tissue culture plates, spinner flasks, aggrewell plates. In some embodiments, the inventors have observed that clusters remain stable in media containing 10% serum. In some embodiments, the conditions that promote clustering include a low serum medium.

It is understood that the cells contacted with a differentiation medium and/or agent can also be simultaneously or subsequently contacted with another agent, such as a growth factor or other differentiation agent or environments to stabilize the cells, or to differentiate the cells further.

Similarly, at least one embryoid body or a precursor thereof can be contacted with at least one differentiation medium or agent and then contacted with at least another differentiation medium or agent. In some embodiments, the cells are contacted with at least one differentiation medium or agent, and the contact is temporally separated, and in some embodiments, cells are contacted with at least one differentiation medium substantially simultaneously. In some embodiments, the cells are contacted with at least two, at least three, at least four, at least five, at least six, at least seven, at least eight, at least nine, or at least ten differentiation mediums or agents.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a neural cell or neural tissue described herein.

The terms “feeder cells” or “feeders” refer to cells of one type that are co-cultured with cells of another type, to provide an environment in which the cells of the second type can grow. The feeder cells are optionally from a different species as the cells they are supporting. In some aspects, a culture or cell population may be referred to as “feeder free”, meaning the composition is essentially free of feeder cells.

The term “growth environment” refers to an environment in which cells of interest will proliferate or differentiate in vitro. Features of the environment include the medium in which the cells are cultured, the temperature, the partial pressure of 02 and CO₂, and a supporting structure (such as a substrate on a solid surface) if present.

The term “nutrient medium” refers to a medium for culturing cells containing nutrients that promote proliferation. The nutrient medium may contain any of the following in an appropriate combination: isotonic saline, buffer, amino acids, antibiotics, serum or serum replacement, and exogenously added factors. A “conditioned medium” is prepared by culturing a first population of cells in a medium, and then harvesting the medium. The conditioned medium (along with anything secreted into the medium by the cells) may then be used to support the growth of a second population of cells.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “expression” refers to the cellular processes involved in producing RNA and proteins and as appropriate, secreting proteins, including where applicable, but not limited to, transcription, translation, folding, modification and processing. “Expression products” include RNA transcribed from a gene and polypeptides obtained by translation of mRNA transcribed from a gene.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, (a) end modifications, e.g., 5′ end modifications (phosphorylation dephosphorylation, conjugation, inverted linkages, etc.) or 3′ end modifications (conjugation, DNA nucleotides, inverted linkages, etc.); (b) base modifications, e.g., replacement with modified bases, stabilizing bases, destabilizing bases, bases that base pair with an expanded repertoire of partners, or conjugated bases; (c) sugar modifications (e.g., at the 2′ position or 4′ position) or replacement of the sugar; and (d) internucleoside linkage modifications, including modification or replacement of the phosphodiester linkages. To the extent that such modifications interfere with translation, the modification is not suitable for the methods and compositions described herein.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. ScL USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having world-wide web address of: “ncbi.nlm nih.gov” for these programs. In a specific embodiment, percent identity is calculated using BLAST2 with default parameters as provided by the NCBI.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, to refer to the ability of stem cells to renew themselves by dividing into the same non-specialized cell type over long periods (e.g., over many months to years). In some instances, proliferation refers to the expansion of cells by the repeated division of single cells into two identical daughter cells.

The term “lineages” as used herein describes a cell with a common ancestry or cells with a common developmental fate. For example, in the context of a cell that is of ectoderm origin or is “ectodermal linage” this means the cell was derived from an ectoderm cell and can differentiate along the ectoderm lineage restricted pathways.

As used herein, the term “xenogeneic” refers to cells that are derived from different species.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

As used herein, the term “DNA” is defined as deoxyribonucleic acid.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “functional fragments” as used herein is a polypeptide having amino acid sequence which is smaller in size than, but substantially homologous to the polypeptide it is a fragment of, and where the functional fragment polypeptide sequence is about at least 50%, or 60% or 70% or 80% or 90% or 100% or greater than 100%, for example 1.5-fold, 2-fold, 3-fold, 4-fold or greater than 4-fold effective biological action as the polypeptide from which it is a fragment of. Functional fragment polypeptides may have additional functions that can include decreased antigenicity, increased DNA binding (as in transcription factors), or altered RNA binding (as in regulating RNA stability or degradation).

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

As used herein, the term “transcription factor” refers to a protein that binds to specific parts of DNA using DNA binding domains and is part of the system that controls the transfer (or transcription) of genetic information from DNA to RNA. As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene. In some cases, expression of the reporter gene may be determined in live cells. Where live cell reporter gene assays are used, reporter gene expression may be monitored at multiple time points, e.g., 2, 3, 4, 5, 6, 8, or 10 or more time points. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The terms “non-human animals” and “non-human mammals” as used herein interchangeably, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

The terms “treating” and “treatment” refer to administering to a subject an effective amount of a composition so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

The term “test compound” refers to any of a small molecule, nucleic acid, amino acid, polypeptide, antibody and antibody-like molecules, aptamers, macrocycles, or other molecules. In certain embodiments, a test compound is a small organic molecule. In one aspect of these embodiments, the small organic molecule has a molecular weight of less than about 5,000 daltons. In certain embodiments, the test compound is other than an amino acid. In other embodiments, the small molecule is other than leucine, isoleucine or analogs of either of the foregoing.

As used herein, “neuro disorder” or “neuro disease” refer to neurodegenerative disorders, neuropsychiatric disorders and/or neurodevelopmental disorders. “Neuro disease” also refers to neurological, neuropsychological, neuropsychiatric, neurodegenerative, or neuropsychopharmacological diseases. Neuro disorders may be any disease affecting neuronal network connectivity, synaptic function and activity. “Neurodegenerative disorder” refers to a disease condition involving neural loss mediated or characterized at least partially by at least one of deterioration of neural stem cells and/or progenitor cells. Non-limiting examples of neurodegenerative disorders include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barré syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, and Tabes dorsalis.

In certain contexts, neurodegenerative disorders encompass neurological injuries or damages to the CNS or the PNS associated with physical injury (e.g., head trauma, mild to severe traumatic brain injury (TBI), spinal cord injury, diffuse axonal injury, craniocerebral trauma, cranial nerve injuries, cerebral contusion, intracerebral haemorrhage and acute brain swelling), ischemia (e.g., resulting from spinal cord infarction or ischemia, ischemic infarction, stroke, cardiac insufficiency or arrest, atherosclerotic thrombosis, ruptured aneurysm, embolism or hemorrhage), certain medical procedures or exposure to biological or chemic toxins or poisons (e.g., surgery, coronary artery bypass graft (CABG), electroconvulsive therapy, radiation therapy, chemotherapy, anti-neoplastic drugs, immunosuppressive agents, psychoactive, sedative or hypnotic drugs, alcohol, bacterial or industrial toxins, plant poisons, and venomous bites and stings), tumors (e.g., CNS metastasis, intraaxial tumors, primary CNS lymphomas, germ cell tumors, infiltrating and localized gliomas, fibrillary astrocytomas, oligodendrogliomas, ependymomas, pleomorphic xanthoastrocytomas, pilocytic astrocytomas, extraaxial brain tumors, meningiomas, schwannomas, neurofibromas, pituitary tumors, and mesenchymal tumors of the skull, spine and dura matter), infections (e.g., bacterial, viral, fungal, parasitic or other origin is selected from the group consisting of pyrogenic infections, meningitis, tuberculosis, syphilis, encephalomyelitis and leptomeningitis), metabolic or nutritional disorders (e.g., glycogen storage diseases, acid lipase diseases, Wemicke's or Marchiafava-Bignami's disease, Lesch-Nyhan syndrome, Farber's disease, gangliosidoses, vitamin B12 and folic acid deficiency), cognition or mood disorders (e.g., learning or memory disorder, bipolar disorders and depression), and various medical conditions associated with neural damage or destruction (e.g., asphyxia, prematurity in infants, perinatal distress, gaseous intoxication for instance from carbon monoxide or ammonia, coma, hypoglycaemia, dementia, epilepsy and hypertensive crises).

“Neuropsychiatric disorder” encompasses mental disorders attributable to diseases of the nervous system. Non-limiting examples of neuropsychiatric disorders include addictions, childhood developmental disorders, eating disorders, degenerative diseases, mood disorders, neurotic disorders, psychosis, sleep disorders, depression, obsessive-compulsive disorder, schizophrenia, visual hallucination, auditory hallucination, eating disorder, bipolar disorder, epilepsy, autism spectrum disorder (ASD), and amyotrophic lateral sclerosis (ALS).

The term “tissue” refers to a group or layer of specialized cells which together perform certain special functions. The term “tissue-specific” refers to a source of cells from a specific tissue.

The terms “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

Stem Cells

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells for producing neural tissues or precursors thereof, germ cells may be used in place of, or with, the stem cells to provide at least one cerebral organoid, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (available on the World Wide Web at subdomain stemcells.nih.gov/info/scireport/2006report.htm, 2006).

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1:Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al., Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase 0200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which are incorporated herein in their entirety by reference.

Briefly, genital ridges processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 1011M forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad γ-irradiation \tilde{ }0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g. a cell not committed to a specific linage) prior to exposure to at least one differentiation medium and/or agent according to the methods as disclosed herein, whereas in other examples it may be desirable to differentiate the stem cells to one or more intermediate cell types prior to exposure of the at least one differentiation medium or agent described herein. For example, the stems cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature, insulin positive cells did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subject's adipose tissue, for example using the CELUTION™ SYSTEM from Cytori, as disclosed in U.S. Pat. Nos. 7,390,484 and 7,429,488 which are incorporated herein in their entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic microenvironment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies and are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

Cloning and Cell Culture

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, or described generally, in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM β-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Scientists at Geron have discovered that pluripotent SCs can be maintained in an undifferentiated state even without feeder cells. The environment for feeder-free cultures includes a suitable culture substrate, particularly an extracellular matrix such as MATRIGEL® or laminin. Typically, enzymatic digestion is halted before cells become completely dispersed (about 5 min with collagenase IV). Clumps of about 10 to 2,000 cells are then plated directly onto the substrate without further dispersal.

Feeder-free cultures are supported by a nutrient medium containing factors that support proliferation of the cells without differentiation. Such factors may be introduced into the medium by culturing the medium with cells secreting such factors, such as irradiated (about 4,000 rad) primary mouse embryonic fibroblasts, telomerized mouse fibroblasts, or fibroblast-like cells derived from pPS cells. Medium can be conditioned by plating the feeders at a density of about 5-6×10⁴ cm⁻² in a serum free medium such as KO DMEM supplemented with 20% serum replacement and 4 ng/mL bFGF. Medium that has been conditioned for 1-2 days is supplemented with further bFGF, and used to support pluripotent SC culture for 1-2 days. Features of the feeder-free culture method are further discussed in International Patent Publication WO 01/51616; and Xu et al., Nat. Biotechnol. 19:971, 2001.

Under the microscope, ES cells appear with high nuclear/cytoplasmic ratios, prominent nucleoli, and compact colony formation with poorly discernable cell junctions. Primate ES cells express stage-specific embryonic antigens (SSEA) 3 and 4, and markers detectable using antibodies designated Tra-1-60 and Tra-1-81 (Thomson et al., Science 282:1145, 1998). Mouse ES cells can be used as a positive control for SSEA-1, and as a negative control for SSEA-4, Tra-1-60, and Tra-1-81. SSEA-4 is consistently present in human embryonal carcinoma (hEC) cells. Differentiation of pluripotent SCs in vitro results in the loss of SSEA-4, Tra-1-60, and Tra-1-81 expression, and increased expression of SSEA-1, which is also found on undifferentiated hEG cells.

Neural Tissue

In some embodiments, the disclosure provides tissue that is or resembles neural or cerebral tissue. In some aspects, the neural tissue includes whole-brain organoids (e.g., cerebral organoids). In certain aspects, the cerebral or brain organoid contains differentiated human stem cells that include one or more types of neurons or cells (e.g., various retina cell types and diverse classes of neurons and progenitors of the cerebral cortex). For example, organoids generate a broad diversity of cells, including subpopulations of neurons and progenitors of the cerebral cortex (e.g., neuronal genes, interneurons, glia cells, forebrain cells, hindbrain cells, midbrain cells, forebrain excitatory neurons, corticofugal projection neurons, callosal projection neurons, TH+ neurons that are necessary for neural network circuits, and the like), as well as retinal cell types (e.g., cortical neurons, subcortical neurons, sensory cells, Muller glial cells, canonical pigmented epithelial cells, photoreceptors, retinal ganglion cells, bipolar cells, amacrine cells, and the like).

In some embodiments, the methods of the invention allow for the generation of cerebral organoids that exhibit one or more features. In certain embodiments, the one or more features include a high degree of cellular heterogeneity and differentiation (e.g., increased diversity), a long survival time (e.g., greater than 10, 11, 12, 13 or more months), neuronal maturity, and display of discrete brain regions (e.g., a mature retina with functional photoreceptors). The cerebral organoids of the invention may be cultured for any period of time. For example, the organoids may be cultured for about 1, 2, 3, 4, 5, 6, 7, 8, 9, 10, 11, 12, 13, 14, 15, 16, 17, 18, 19, 20, 21, 22, 23 or 24 months.

In some aspects, mature cells of the cerebral organoids (e.g., those cells that have experienced progressive maturation) may exhibit structural traits typical of more mature neurons. Cells of the cerebral organoids may be examined at varying time points over the course of organoid development (e.g., at 1, 2, 3, 4, 5, 6, 7, 8, 9 and/or months) for signs of maturation, including development of structural traits and expression of various markers. For example, mature cells may exhibit the formation of synapses and the presence of dendritic spines. In some aspects, long-term cultures of human whole-brain organoids may develop spontaneously active neurons and neuronal networks. In addition, certain markers may emerge before other markers during maturation of the organoids. For example, neural progenitor markers may emerge before pan-neuronal markers and markers for other neuronal identities (e.g., markers for glutamatergic, GABAergi, and dopaminergic neuronal identities). In some aspects, mature organoids (e.g., organoids that have matured for six or more months) contain additional clusters not present at earlier time points (e.g., at 3 months).

In other embodiments, the methods of the invention generate mature retinal tissue. Retinal cells of the cerebral organoids may be examined at varying time points over the course of organoid development (e.g., at 1, 2, 3, 4, 5, 6, 7, 8, 9 and/or 10 months) for signs of maturation, including development of structural traits and expression of various markers. In some embodiments, the mature retinal tissue has photoreceptors that are normally present only in the last stages of retinal tissue differentiation. In some embodiments, long-term cultures of photoreceptor-like cells generate photoreceptor-like cells that respond to physiological stimuli (e.g., cells that are responsive to non-invasive, light based sensory stimulation). In addition, certain markers may emerge before other markers during maturation of the retinal cells. In some embodiments, key genes involved in phototransduction at advanced states of differentiation are expressed. In some aspects, photoreceptors progressively mature within organoids between 3 and 6 months.

In some embodiments, other functional sensory cells that are generated include, but are not limited to, one or more of functional photoreceptors (e.g., eye cells), functional auditory receptors, functional olfactory receptors, functional taste receptors, and functional touch receptors.

In some embodiments, the tissue that is or resembles neural or cerebral tissue comprises substantially all cells found in the brain or progenitors thereof. One can use any means common to one of ordinary skill in the art to confirm the presence of neural tissue produced by the differentiation of at least neuroectodermal cells or precursors thereof by exposure to at least one differentiation medium or agent as described herein. In some embodiments, such cells or tissue can be identified by selective gene expression markers. In some embodiments, the method can include detecting the positive expression (e.g. the presence) of a marker for neural tissue or cells. In some embodiments, the marker can be detected using a reagent. A reagent for a marker can be, for example, an antibody against the marker or primers for a RT-PCR or PCR reaction, e.g., a semi-quantitative or quantitative RT-PCR or PCR reaction. Such markers can be used to evaluate whether a neural tissue or cell has been produced. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The progression of at least one neuroectodermal cell or precursor thereof to a neural tissue can be monitored by determining the expression of markers characteristic of neural tissue. In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of neural tissue, as well as the lack of significant expression of markers characteristic of the neuroectodermal cells or precursors thereof, e.g., pluripotent stem cell or ectodermal cell from which it was derived is determined.

As described in connection with monitoring the production of neural tissue from a neuroectodermal cell, qualitative or semi-quantitative techniques, such as blot transfer methods and immunocytochemistry, can be used to measure marker expression, using methods commonly known to persons of ordinary skill in the art. Alternatively, marker expression can be accurately quantitated through the use of technique such as quantitative-PCR by methods ordinarily known in the art.

In some embodiments, cells of the inventive culture express one or more gene expression markers selected from forebrain markers BF1 and Six3. Alternatively, or in addition, cells of the inventive culture express one or more gene expression markers selected from hindbrain markers Krox20 and IIs1. At a certain stage of development forebrain markers are expressed in increased amounts as compared to hindbrain markers in the tissue.

In some embodiments, the cerebral tissue culture can alternatively or in addition be characterized by comprising cells expressing one or more markers selected from Pax6, Sox2, Tbr2, DCX, Foxg1, Prox1, Fzd9, Nkx2.1, Reelin, Tbr1, Ctip2, Satb2, TH, opsin, s100beta, Dkk3, SOX9, MLANA, MITF, CRX, RCVN, NRL, NEFL, NEFM, LHX4, PCP2, TFAP2B, SLC32A1, GNAT1, PDE6A, PDE6B, ROM1, EOMES, ELAV4, GAD1, GAD2, MAP2, GAP43, EBF1, Otx2, Gbx2, AQP4, GFAP, OLIG2, VGLUT1, GABA, MYH3, MYH8, MYL1, MYLPF, TOP2A, MKI67, BF1, Six3, Krox20, Ils1 or any combination thereof. In some aspects, cells express one or more markers indicative of retinal cells. In other aspects, cells express one or more markers indicative of neural or cerebral cells. These markers may be expressed during any stage of the culture during the described methods, and may be expressed in the provided tissue culture.

In certain embodiments, the cerebral tissue culture comprises cells, which express FoxG1. FoxG1 is expressed in cells of telencephalic identity. In certain embodiments, the cerebral tissue culture comprises cells, which express Brn2. Brn2 is expressed in one or more neuronal subtypes including cells localized in the upper layers of the cerebral cortex. In certain embodiments, the cerebral tissue culture comprises cells, which express Satb2. Satb2 is expressed in one or more neuronal subtypes including callosal projection neurons localized in the upper layers of the cerebral cortex. In certain embodiments, the cerebral tissue culture comprises cells, which express Ctip2. Ctip2 is expressed in one or more neuronal subtypes including cells localized in the deep layers of the cerebral cortex. In certain embodiments, the cerebral tissue culture comprises cells, which express calretinin. Calretinin is expressed in one or more neuronal subtypes including GABAergic cortical interneurons. In certain embodiments, the cerebral tissue culture comprises cells, which express Pax6. Pax6 is expressed in radial glia/NSCs cells. Pax6 acts as a marker for neural progenitors. In certain embodiments, the cerebral tissue culture comprises cells, which express Sox2. Sox2 is expressed in radial glia/NSCs cells. In certain embodiments, the cerebral tissue culture comprises cells, which express Tbr2. Tbr2 is expressed in intermediate cortical progenitor cells. In certain embodiments, DCX is expressed in differentiating neuronal cells. In certain embodiments, the cerebral tissue culture comprises cells, which express Prox1. Prox1 is expressed in one or more neuronal subtypes including hippocampal neurons. In certain embodiments, the cerebral tissue culture comprises cells, which express Fzd9. Fzd9 is expressed in the brain and the eyes, as well as in neural precursor cells. In certain embodiments, the cerebral tissue culture comprises cells, which express Reelin. Reelin is expressed in a number of neuronal subtypes including Cajal-retzius cells. In certain embodiments, the cerebral tissue culture comprises cells, which express Tbr1. Tbr1 is expressed in one or more neuronal subtypes including deep-layer cortical neurons. In certain embodiments, the cerebral tissue culture comprises cells, which express Cux1. Cux1 is expressed in one or more neuronal subtypes including cells localized in the upper layers of the cerebral cortex. In certain embodiments, the cerebral tissue culture comprises cells, which express the marker opsin. Opsins are expressed in one or more neuronal subtypes including photoreceptors localized in the retina. In certain embodiments, the cerebral tissue culture comprises cells, which express TH. TH is a marker of dopaminergic neurons. In certain embodiments, the cerebral tissue culture comprises cells, which express s100 beta. s100 beta is expressed in glial cells including astrocytes. In certain embodiments, the cerebral tissue culture comprises cells, which express Nkx2.1 Nkx2.1 acts as a marker of neural progenitors.

In certain embodiments, the cerebral tissue culture comprises cells which express Dkk3. In some embodiments, the cerebral tissue culture comprises cells which express SOX9. Dkk3 and SOX9 may act as markers for Muller glia cells. In certain embodiments, the cerebral tissue culture comprises cells which express MLANA. In some embodiments, the cerebral tissue culture comprises cells which express MITF. MLANA and MITF may act as markers for canonical pigmented epithelial cells. In certain embodiments, the cerebral tissue culture comprises cells which express CRX. In some embodiments, the cerebral tissue culture comprises cells which express RCVN. In certain embodiments, the cerebral tissue culture comprises cells which express NRL. CRX, RCVN and NRL may act as photoreceptor markers. In some embodiments, the cerebral tissue culture comprises cells which express NEFL. In certain embodiments, the cerebral tissue culture comprises cells which express NEFM. NEFL and NEFM may act as markers for retinal ganglion cells. In some embodiments, the cerebral tissue culture comprises cells which express LHX4. In certain embodiments, the cerebral tissue culture comprises cells which express PCP2. LHX4 and PCP2 may act as markers for bipolar cells. In some embodiments, the cerebral tissue culture comprises cells which express TFAP2B. In certain embodiments, the cerebral tissue culture comprises cells which express SLC32A1. TFAP2B and SLC32A1 may act as markers for amacrine cells.

In some embodiments, the cerebral tissue culture comprises cells which express GNAT1. GNAT1 may act as a marker for the alpha subunit of rod transducin. In certain embodiments, the cerebral tissue culture comprises cells which express PDE6A. In some embodiments, the cerebral tissue culture comprises cells which express PDE6B. PDE6A and PDE6B may act as markers for the alpha and beta subunits of the rod cGMP-phosphodiesterase. In some embodiments, the cerebral tissue culture comprises cells which express ROM1. ROM1 may act as a marker for the rod outer membrane protein.

In certain embodiments, the cerebral tissue culture comprises cells which express EOMES. In some embodiments, the cerebral tissue culture comprises cells which express ELAV4. EOMES and ELAV4 may act as markers for canonical genes for intermediate progenitors. In certain embodiments, the cerebral tissue culture comprises cells which express GAD1. In some embodiments, the cerebral tissue culture comprises cells which express GAD2. GAD1 and GAD2 may act as markers for interneurons.

In certain embodiments, the cerebral tissue culture comprises cells which express MAP2. In some embodiments, the cerebral tissue culture comprises cells which express GAP43. In some embodiments, the cerebral tissue culture comprises cells which express Otx2. In certain embodiments, the cerebral tissue culture comprises cells which express Gbx2. In some embodiments, the cerebral tissue culture comprises cells which express OLIG2. In certain embodiments, the cerebral tissue culture comprises cells which express VGLUT1. In some embodiments, the cerebral tissue culture comprises cells which express GABA. MAP2 and GABA act as markers for neurons. MAP2 may act as a pan-neuronal marker and GABA may act as a marker for GABAergic neuronal identity. Vglut1 acts as a marker for glutamatergic neuronal identity and TH may act as a marker for dopaminergic neuronal identity. GFAP and OLIG2 act as markers for glial cells. Nkx2.1, Otx2 and Gbx2 act as markers for neural progenitors. In addition, Nkx2.1 and Pax6 act as a marker for germinal zones of the forebrain, Otx2 acts as a marker for germinal zones of the midbrain, and Gbx2 acts as a marker for germinal zones of the hindbrain.

In some embodiments, the cerebral tissue culture comprises cells which express AQP4. In certain embodiments, the cerebral tissue culture comprises cells which express GFAP. AQP4 and GFAP may act as canonical astrocyte markers. In certain embodiments, the cerebral tissue culture comprises cells which express TOP2A. In some embodiments, the cerebral tissue culture comprises cells which express MKI67. TOP2A and MKI67 may act as markers of highly proliferative progenitors. In certain embodiments, the cerebral tissue culture comprises cells which express EBF1. TH and EBF1 may act as dopaminergic markers.

In some embodiments, the cerebral tissue culture comprises cells which express Myogenin. In certain embodiments, the cerebral tissue culture comprises cells which express MYH3. In some embodiments, the cerebral tissue culture comprises cells which express MYH8. In certain embodiments, the cerebral tissue culture comprises cells which express MYL1. In some embodiments, the cerebral tissue culture comprises cells which express MYLPF. Mesodermal markers may be expressed for muscle-specific genes (Myogenin) and various Myosin genes (MYH3, MYH8, MYL1 and MYLPF).

It is understood that the present invention is not limited to those markers listed as neural tissue markers herein, and the present invention also encompasses markers such as cell surface markers, antigens, and other gene products including ESTs, RNA (including microRNAs and antisense RNA), DNA (including genes and cDNAs), and portions thereof.

In some embodiments, the cerebral tissue culture further comprises a step of co-culturing with any other cell type. In some embodiments, the co-cultured cells are one or more of microglia, oligodendrocytes, endothelial cells, cells of the immune system and stromal cells. In some embodiments, the cerebral tissue culture comprises a cerebral organoid co-cultured with microglia or other cell types to model disease. In some embodiments, the co-culture of cerebral organoid and microglia is a model of synaptic pruning affected by psychiatric disease.

Generating Neural Tissue

Aspects of the disclosure relate to generating neural tissue (e.g., cerebral organoids). Generally, the neural tissue produced according to the methods disclosed herein can comprise a mixture or combination of different cells, e.g., for example a mixture of neural or cerebral cells, such as retinal or cortical organoids, other organoids, such as auditory organoids, and/or other pluripotent or stem cells.

The neural tissue can be produced according to any suitable culturing protocol to differentiate a stem cell or pluripotent cell to a desired stage of differentiation. In some embodiments, the at least one stem cell or pluripotent cell is a human cell. In some embodiments, the at least one stem cell or pluripotent cell is not a human cell. In certain aspects, the at least one stem cell or pluripotent cell is a mouse cell. In some embodiments, the neural tissue is produced by culturing at least one stem cell for a period of time and under conditions suitable for the at least one stem cell to differentiate into the neural tissue or a precursor thereof.

In some embodiments, the neural tissue or precursor thereof is a substantially pure population of neural tissue or precursors thereof. In some embodiments, a population of neural tissue or precursors thereof comprises a mixture of pluripotent cells or differentiated cells. In some embodiments, a population of neural tissue or precursors thereof is substantially free or devoid of embryonic stem cells or pluripotent cells.

In some embodiments, the neural tissue or precursors thereof are maintained in culture by methods known by one of ordinary skill in the art, and in some embodiments, propagated prior to being converted into neural tissue by the methods as disclosed herein.

In some embodiments, embryoid bodies are generated from stem cells or pluripotent cells. In certain embodiments, the embryoid bodies are differentiated to form neural or cerebral tissue. In some embodiments, embryoid bodies are subject to germ layer differentiation. The embryoid bodies may then be treated with neural induction medium causing the embryoid bodies to indicate neuroectodermal differentiation. In certain embodiments, neuroepithelial tissue develops as a result of differentiation. The neuroeptihelial tissue may be treated with cerebral organoid differentiation medium to form organoids. The resulting organoids may be grown and maintained in a tissue culture incubator (e.g., an orbital shaker, a spinning bioreactor, a spinner flask, etc.). In some embodiments, the speed of the orbital shaker is optimized to allow for long term growth of the organoids and development of organoids with sensory perception and response. In some embodiments, the speed of the spinning bioreactor or spinner flask is optimized to allow for long term growth of the organoids and development of organoids with sensory perception and response. In some aspects, the rotation rate of the orbital shaker or spinner flask is about 25-150 rpm. In some aspects, the rotation rate of the orbital shaker or spinner flask is about 50-100 rpm. In some aspects, the rotation rate of the orbital shaker or spinner flask is about 50-75 rpm. In some aspects, the rotation rate of the orbital shaker or spinner flask is about 60 rpm. In some aspects, the rotation rate of the orbital shaker or spinner flask is about 5 RPM, 10 RPM, 15 RPM, 20 RPM, 25 RPM, 30 RPM, 35 RPM, 40 RPM, 45 RPM, 50 RPM, 55 RPM, 60 RPM, 65 RPM, 70 RPM, 75 RPM, 80 RPM, 85 RPM, 90 RPM, 95 RPM, 100 RPM, 105 RPM, 110 RPM, 115 RPM, 120 RPM, 125 RPM, 130 RPM, 135 RPM, 140 RPM, 145 RPM, or 150 RPM. In some aspects, the rotation rate of the orbital shaker or spinner flask is a rate that allows sufficient oxygen diffusion in the medium and at the same time preserves the integrity of the brain organoids. In some aspects, the rotation rate of the orbital shaker or spinner flask that allows enough oxygen diffusion in the medium and at the same time preserves the integrity of the brain organoids is about 50-75 rpm, preferably about 60 rpm.

Detailed protocols for generating neural tissue, include, but are not limited to, those described in Lancaster, et al. Nature Protocols, 9.10:2329 (2014) and U.S. Patent Application Nos. 62/273,819 (filed Dec. 31, 2015) and 62/342,566 (filed May 27, 2016), which are incorporated herein by reference. In some embodiments, the detailed protocols may be modified. For example, the protocol detailed by Lancaster may be modified in one or more instances.

In some embodiments, 2,000, or in other embodiments 2,500 feeder-independent human embryonic live stem cells were plated for formation of embryoid bodies. The cells may adhere to the plates while forming embryoid bodies. BDNF may be added to the final differentiation medium. In some aspects, the addition of BDNF to the final differentiation medium allows the culture period to be prolonged, thereby limiting premature cell death and enabling long-term, progressive development for 9-13 months. The resulting method produces embryoid bodies that progress to form long-term organoid cultures (e.g., greater than 9 months). The percentage of embryoid bodies that progressed was greater than 95% when using an iPSC11a cell line. The percentage of embryoid bodies that progressed was about 70% when using a HuES66 cell line.

A high percentage of the embryoid bodies formed following the methods of the invention exhibit smooth edges and show a clear and bright surface indicative of neuroectoderermal germ layer differentiation.

In some embodiments, during the initial neural induction step, the embryoid bodies are treated with a combination of hESC medium and neural induction medium at a 1:3 ratio, as compared to only a neural induction medium as described by Lancaster. A gradual switch from hESC medium to neural induction medium causes 60-100% of the embryoid bodies to form neuroepithelial buds when placed in Matrigel droplets. In some embodiments, after the embryoid bodies had been embedded in Matrigel and grown in cerebral organoid differentiation medium for at least one month, the differentiation medium was switched to a modified cerebral organoid differentiation medium containing 100 ng/ml of the neurotrophin BDNF. The methods of the invention allow for the long term culture and differentiation of the organoids.

In some embodiments, three dimensional neural tissue compositions are generated by the methods disclosed herein and one or more cell types are isolated from the compositions. The organoids are used as “bio-incubators” to generate cells that can be isolated and printed in any desired conformation and/or combination. The isolated cells may include subpopulations of neurons and progenitors of the cerebral cortex (e.g., neuronal genes, interneurons, glia cells, forebrain cells, hindbrain cells, midbrain cells, forebrain excitatory neurons, corticofugal projection neurons, callosal projection neurons, TH+ neurons that are necessary for neural network circuits, and the like), as well as retinal cell types (e.g., cortical neurons, subcortical neurons, sensory cells, Muller glial cells, canonical pigmented epithelial cells, photoreceptors, retinal ganglion cells, bipolar cells, amacrine cells, and the like). In some embodiments, the isolated one or more cell types are used for screening for cell-cell interactions and/or neural network properties. In some embodiments, the isolated cells comprise one or more retinal cell types including, but not limited to, mature retinal cell types. The isolated retinal cell types can be further cultured on a patch or other suitable cell substrate.

In some embodiments, neural tissue generated by the methods disclosed herein is co-cultured with another cell type. In some embodiments, the cell type is one or more of microglia, oligodendrocytes, endothelial cells, cells of the immune system and stromal cells. In specific embodiments, cerebral organoids are produced by the methods herein and then co-cultured with another cell type. In some embodiments, the cerebral organoids are co-cultured with microglia. The microglia or other cell type may be transplanted into the cerebral organoid or three dimensional neural tissue.

Brain Organoid-Machine Interface

In some embodiments, the present invention provides methods to record and probe neural network circuit activity in brain organoids and for the generation of the first “brain organoid-machine interface” (BOMI) to assay for higher-order circuit function such as sensory-response learning. In some aspects, a brain organoid is interfaced with a computer, whereby spontaneous sensory circuit activity input is analyzed and the software outputs instructions to the stimulus generating device in a loop mimicking sensory-response learning.

In some embodiments, the invention provides methods to measure one or more parameters from a brain organoid. The brain organoid may be from a long-term organoid culture (e.g., a culture that is greater than 4 months). In certain embodiments, the brain organoid responds to a sensory stimulus. In some embodiments, one or more parameters are measured in response to a sensory stimulus. In certain aspects, the one or more parameters include: a) neural (monosynaptic) and/or network (polysnaptic) evoked response; b) individual spike trains (e.g., spike delays, peri-stimulus event histograms, inter spike interval histograms); c) correlations across spike trains and network synchronization; d) network topology (e.g., vertices, edges, path length, clustering, small-worldliness); and/or e) selective pharmacology to study basic physiology (e.g., dissect neural networks) and disease models. For example, spikes (e.g., action potentials) may be isolated in organoids (e.g., organoids that are older than 8 months). In some aspects, the presence of distinct firing patterns is identified in the neurons sampled.

At a basic level these circuits can be stimulated in a way that allows them to “learn” how to respond to applied stimuli. In some embodiments, this “training” process can be controlled by a computer interface that decodes circuit response and instruct closed-loop feedback stimulation.

In some aspects, light driven physiological input modifies the state of organoid neural network. Specifically, light stimulus evoked polysynaptic activity has been confirmed by spike time latency, sensitivity to calcium free aCSF, and AMPA receptor antagonist administration.

In some embodiments, a brain organoid machine interface comprises a multi-probe electrode array (e.g., a high density silicon microelectrode) configured to collect electrophysiological signals from neural tissue; a first processor connected to the multi-probe electrode array; a second processor coupled to a sensory generating device; and software configured to decode circuit response and instruct feedback stimulation to the sensory generating device. In certain aspects, the first processor is configured to collect and store the electrophysiological signals. In certain aspects, the sensory generating device is a light-emitting diode (LED).

In some embodiments, the sensory generating device generates one or more stimuli selected from the group consisting of a visual stimulus, an auditory stimulus, an olfactory stimulus, a taste stimulus, and a touch stimulus. In some embodiments, the brain organoid machine interface measures the neural tissue for spontaneous activity in response to the stimulus and/or for network activity in response to the stimulus. The majority of recorded spikes may be driven by fast non-NMDA-receptor mediated excitatory synaptic transmission. In some embodiments, the brain organoid machine interface measures performance of the neural tissue during a learning session. A learning session may occur where a brain organoid is interfaced with a computer, whereby spontaneous sensory circuit activity input is analyzed and the software outputs instructions to the stimulus generating device in a loop mimicking sensory-response learning.

In some aspects, a stimulus is a visual (e.g., light) stimulus. Light stimulation may be capable of modulating the activity of light-photosensitive cells that develop within an organoid. Light application may specifically disrupt ongoing firing patterns of light-sensing cells. Light-based, physiological sensory stimuli activate light-sensing cells, thereby modulating downstream neuronal activity within organoids. In some aspects, an activity-dependent gene (e.g., cFos) is upregulated in organoids (e.g., 8 month organoids) exposed to light compared to organoids maintained in the dark.

In each aspect discussed above, the brain organoid machine interface can include brain organoid tissue. In some embodiments, the neural tissue incorporated into the brain organoid-machine interface is generated from patient derived stem cells. The brain organoid machine interface may screen the patient derived neural tissue for dysregulation of spontaneous activity in response to one or more stimuli and/or for defects of network activity in response to one or more stimuli. In some aspects, the brain organoid machine interface screens the patient derived neural tissue for defects in performance of the neural tissue during a learning session.

Modeling of Diseases

The invention also provides methods of modeling diseases involving neural tissue. In some embodiments, the modeling comprises generating brain organoids from induced pluripotent stem cells (iPSCs) derived from a patient with a disease or related disease to be modeled. In some cases, the disease is a neuro disease, but the type of disease is not limited. In some cases, the disease is a neuropsychiatric disease.

In some embodiments, the modeling comprises generating brain organoids by methods disclosed herein and inducing a disease or disease-like state. The disease or disease-like state may be induced through any method known in the art. For example, induction may be via a chemical or biological agent such as a virus, neurotoxin, bacteria, metal, small molecule, peptide or polynucleotide. The brain organoid for modeling may also be produced by any known genetic engineering technique. In some embodiments, the brain organoid may be produced from cells having one or more modified genes or genetic locus. For example, the cells may have one or more genes partially or fully deleted or partially or fully added. The brain organoids used of modeling may be cultured for any period including about 1, 2, 3, 4, 5, 6, 7, 8, 9, 10, 11, 12, 13, 14, 15, 16, 17, 18, 19, 20, 21, 22, 23 or 24 months. In some embodiments, the brain organoid is cultured for about 9 months. In some embodiments, the brain organoid is cultured for about 9-13 months.

In some embodiments, the methods of modeling may include co-culturing the brain organoid with any other cell type. The other cell types include, but are not limited to, one or more of microglia, oligodendrocytes, endothelial cells, cells of the immune system and stromal cells. In specific embodiments, the modeling comprises co-culture of cerebral organoids with microglia or other cell types transplanted into the cerebral organoid. In some embodiments synaptic pruning affected in psychiatric disease are modeled by co-culturing with, for example, microglia transplanted into the organoid.

Screening of Diseases

The invention provides a method of screening patients with a neuro disorder or neuro disease or any disease affecting synaptic function, neuronal network activity and stimulation, through the generation of brain organoids from patient derived induced pluripotent stem cells (iPSCs). In some aspects, organoids are generated from iPSCs genetically engineered to carry mutations associated with one or more neuro disorders. A subject or patient can be one who has been previously diagnosed with or identified as suffering from or having a condition, disease, neuro disease or neuro disorder described herein in need of treatment or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. Rather, a subject can include one who exhibits one or more risk factors or symptoms for a condition or one or more complications related to a condition. A “subject in need” of treatment for a particular condition can be a subject having that condition, diagnosed as having that condition, or at increased risk of developing that condition relative to a given reference population.

In some embodiments, the methods described herein comprise selecting a subject diagnosed with, suspected of having, or at risk of developing a neuro disorder or neuro disease as described herein.

In some aspects, patient derived neural tissue is screened for a neuro disease and/or for pathology of neuronal network connectivity, synaptic function and synaptic activity. In some aspects, the disease may be depression, obsessive-compulsive disorder, schizophrenia, visual hallucination, auditory hallucination, eating disorder, bipolar disorder, epilepsy, autism spectrum disorder (ASD), amyotrophic lateral sclerosis (ALS) and any disease affecting neuronal network connectivity, synaptic function and synaptic activity.

The disclosure contemplates methods in which neural tissue is generated according to the methods described herein from iPS cells derived from cells extracted or isolated from individuals suffering from a disease (e.g., a neuro disease, such as epilepsy, autism spectrum disorder (ASD), schizophrenia, bipolar disorder), and that neural tissue is compared to normal neural tissue from healthy individuals not having the disease to identify differences between the generated neural tissue and normal neural tissue which could be useful as markers for disease (e.g., neuropsychiatric). In some embodiments, the iPS cells and/or neural tissue derived from neuropsychiatric patients are used to screen for agents (e.g., agents which are able to modulate genes contributing to a neurospychiatric phenotype).

The patient derived organoids are screened for dysregulation of spontaneous activity, defects to stimulus induced activity, and also for performance during BOMI learning session. In some aspects, a brain organoid machine interface is utilized to screen for one or more neuro diseases and/or for pathology of neuronal network connectivity, synaptic function and synaptic activity. The brain organoid machine interface may comprise a multi-probe electrode array configured to collect electrophysiological signals from neural tissue, a first processor connected to the multi-probe electrode array; a second processor coupled to a sensory generating device; and software configured to decode circuit response and instruct feedback stimulation to the sensory generating device. In certain aspects, the first processor is configured to collect and store the electrophysiological signals. In some embodiments, the neural tissue incorporated into the brain organoid-machine interface is generated from patient derived stem cells. The brain organoid machine interface may screen the patient derived neural tissue for dysregulation of spontaneous activity in response to one or more stimuli and/or for defects of network activity in response to one or more stimuli. In some aspects, the brain organoid machine interface screens the patient derived neural tissue for defects in performance of the neural tissue during a learning session.

Screening of Agents to Treat a Neuro Disease

In some embodiments, the invention provides a method of screening test agents to identify treatment agents for a neuro disease or diseases affecting neuronal network connectivity, synaptic function and/or synaptic activity. In some aspects, neural tissue exhibiting features of a neuro disease is generated as described by the methods of the invention. The neural tissue may be generated from iPSCs obtained from a patient having a neuro disease (e.g., a neurodegenerative disorder, such as epilepsy, autism spectrum disorder (ASD), bipolar disorder, schizophrenia or a neurological, neuropsychological, neuropsychiatric, neurodegenerative, or neuropsychopharmacological disease). In some embodiments, the neural tissue has functional sensory receptors (e.g. photoreceptors). In certain embodiments, the neural tissue is treated with a test agent. A sensory stimulus may be applied to the treated neural tissue and the results measured and recorded (e.g., using the brain organoid machine interface). The stimulus results of the treated tissue may be compared to control levels. In certain embodiments, stimulus results of the treated neural tissue are similar to the control levels, exhibiting the beneficial effects of the test agent on a neuro disorder. In alternative embodiments, stimulus results of the treated neural tissue are significantly different from the control levels, demonstrating that the test agent does not treat the specific neuro disorder tested.

Compositions and Kits

Described herein are kits for practicing methods disclosed herein and for making neural tissue or cerebral organoids disclosed herein. In one aspect, a kit includes at least one embryoid body or precursor thereof and at least one differentiation medium or agent as described herein, and optionally, the kit can further comprise instructions for converting at least one embryoid body or precursor thereof to a population of cerebral organoids using a method described herein. In some embodiments, the kit comprises at least two differentiation mediums or agents. In some embodiments, the kit comprises at least three differentiation mediums or agents. In some embodiments, the kit comprises at least four differentiation mediums or agents. In some embodiments, the kit comprises at least five differentiation mediums or agents. In some embodiments, the kit comprises differentiation mediums or agents for differentiating pluripotent cells to ectodermal cells. In some embodiments, the kit comprises differentiation mediums or agents for differentiating ectodermal cells to neuroectodermal cells. In some embodiments, the kit comprises differentiation mediums or agents for differentiating neuroectodermal cells to cerebral organoids.

In some embodiments, the kit comprises any combination of differentiation mediums or agents, e.g., for differentiating stem cells to ectodermal cells, differentiating ectodermal cells to neuroectodermal cells, and differentiating neuroectodermal cells to cerebral organoids.

In one embodiment, the kit can comprise a pluripotent stem cell for the purposes of being used as a positive control, for example to assess or monitor the effectiveness or ability of a compound of formula (I) to chemically induce the pluripotent stem cell to differentiate into at least one neuroectodermal cell or precursors thereof, and subsequently into a cerebral organoid. Accordingly, the kit can comprise sufficient amount of at least one differentiation medium or agent for inducing the differentiation of a control pluripotent stem cell population (positive control) as well as inducing the differentiation of a population of pluripotent stem cells of interest (e.g. an iPS cell) into at least one neuroectodermal cell or precursors thereof, or into a cerebral organoid.

In some embodiments, the compound in the kit can be provided in a watertight or gas tight container which in some embodiments is substantially free of other components of the kit. The compound can be supplied in more than one container, e.g., it can be supplied in a container having sufficient reagent for a predetermined number of reactions e.g., 1, 2, 3 or greater number of separate reactions to induce pluripotent stem cells to ectodermal cells, and subsequently into neuroectodermal cells, and subsequently into cerebral organoids. A brain organoid differentiation medium or agent can be provided in any form, e.g., liquid, dried or lyophilized form. It is preferred that a compound(s) (e.g., differentiation medium or agent) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

In some embodiments, the kit further optionally comprises information material. The informational material can be descriptive, instructional, marketing or other material that relates to the methods described herein and/or the use of a compound(s) described herein for the methods described herein.

The informational material of the kits is not limited in its instruction or informative material. In one embodiment, the informational material can include information about production of the compound, molecular weight of the compound, concentration, date of expiration, batch or production site information, and so forth. In one embodiment, the informational material relates to methods for administering the compound. Additionally, the informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In one embodiment, the informational material can include instructions to administer a compound(s) (e.g., a differentiation medium or agent) as described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent, e.g., for inducing pluripotent stem cells (e.g., in vitro) or for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

A differentiation medium or agent as described herein can be provided in any form, e.g., liquid, dried or lyophilized form. It is preferred that a compound(s) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

The kit can include one or more containers for the composition containing at least one differentiation medium or agent as described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The kit can also include a component for the detection of a marker for cerebral organoids, e.g., for a marker described herein, e.g., a reagent for the detection of cerebral organoids. Or in some embodiments, the kit can also comprise reagents for the detection of negative markers of cerebral organoids for the purposes of negative selection of cerebral organoids or for identification of cells which do not express these negative markers (e.g., cerebral organoids). The reagents can be, for example, an antibody against the marker or primers for a RT-PCR or PCR reaction, e.g., a semi-quantitative or quantitative RT-PCR or PCR reaction. Such markers can be used to evaluate whether an iPS cell has been produced. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

It may be desirable to perform an analysis of the karyotype of the cerebral organoids. Accordingly, the kit can include a component for karyotyping, e.g., a probe, a dye, a substrate, an enzyme, an antibody or other useful reagents for preparing a karyotype from a cell.

The kit can include cerebral organoids, e.g., cerebral organoids derived from the same type of neuroectodermal cell or precursor thereof, for example for the use as a positive cell type control.

The kit can also include informational materials, e.g., instructions, for use of two or more of the components included in the kit.

The articles “a”, “an” and “the” as used herein, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. It should also be understood that any embodiment of the invention, e.g., any embodiment found within the prior art, can be explicitly excluded from the claims, regardless of whether the specific exclusion is recited in the specification.

As used herein the term “comprising” or “comprises” is used in reference to compositions, methods, and respective component(s) thereof, that are essential to the invention, yet open to the inclusion of unspecified elements, whether essential or not. The term “consisting essentially of” refers to those elements required for a given embodiment. The term permits the presence of additional elements that do not materially affect the basic and novel or functional characteristic(s) of that embodiment of the invention. The term “consisting of” refers to compositions, methods, and respective components thereof as described herein, which are exclusive of any element not recited in that description of the embodiment.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% of a number in either direction (greater than or less than the number) unless otherwise stated or otherwise evident from the context (except where such number would impermissibly exceed 100% of a possible value).

Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group.

Certain claims are presented in dependent form for the sake of convenience, but any dependent claim may be rewritten in independent format to include the limitations of the independent claim and any other claim(s) on which such claim depends, and such rewritten claim is to be considered equivalent in all respects to the dependent claim (either amended or unamended) prior to being rewritten in independent format. It should also be understood that, unless clearly indicated to the contrary, in any methods claimed herein that include more than one act, the order of the acts of the method is not necessarily limited to the order in which the acts of the method are recited, but the invention includes embodiments in which the order is so limited. It is contemplated that all aspects described above are applicable to all different embodiments of the invention. It is also contemplated that any of the above embodiments can be freely combined with one or more other such embodiments whenever appropriate.

### EXAMPLES

### Example 1—Protracted In Vitro Development of Human Whole-Brain Organoids

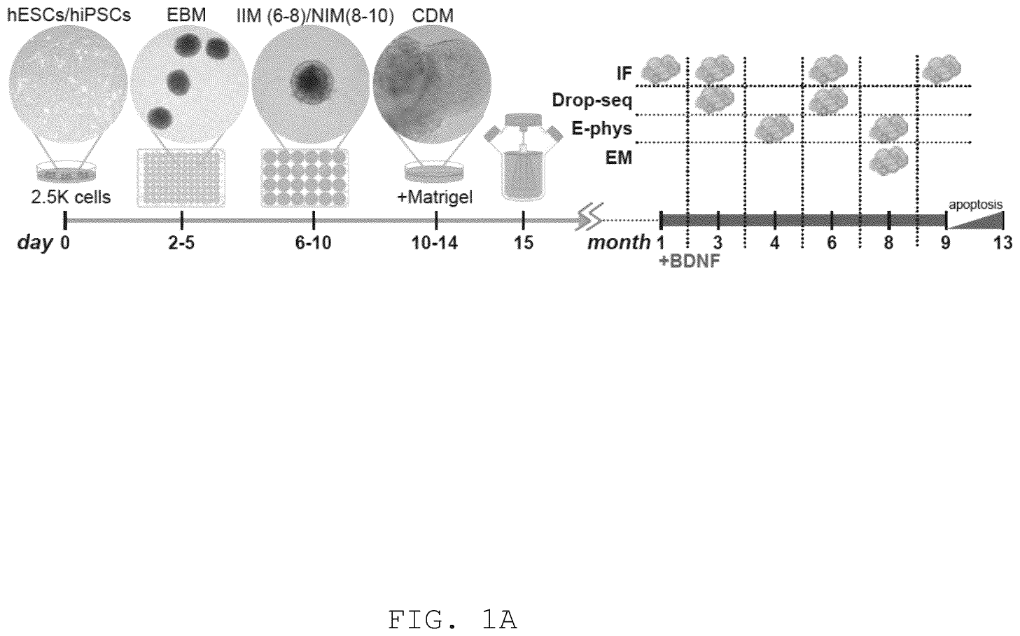

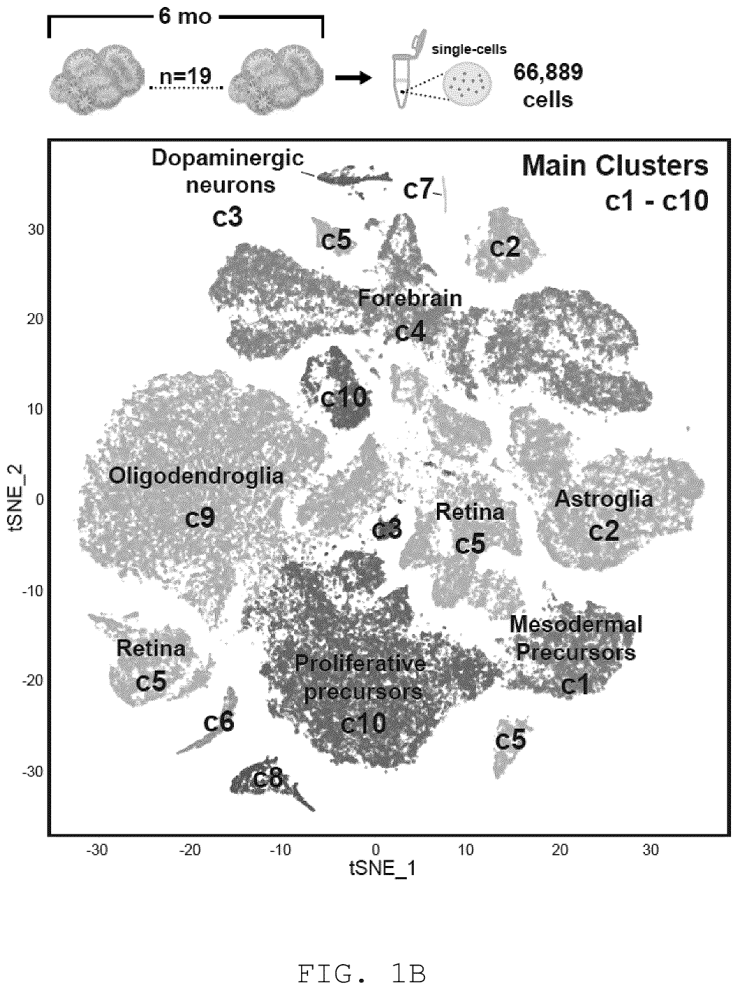

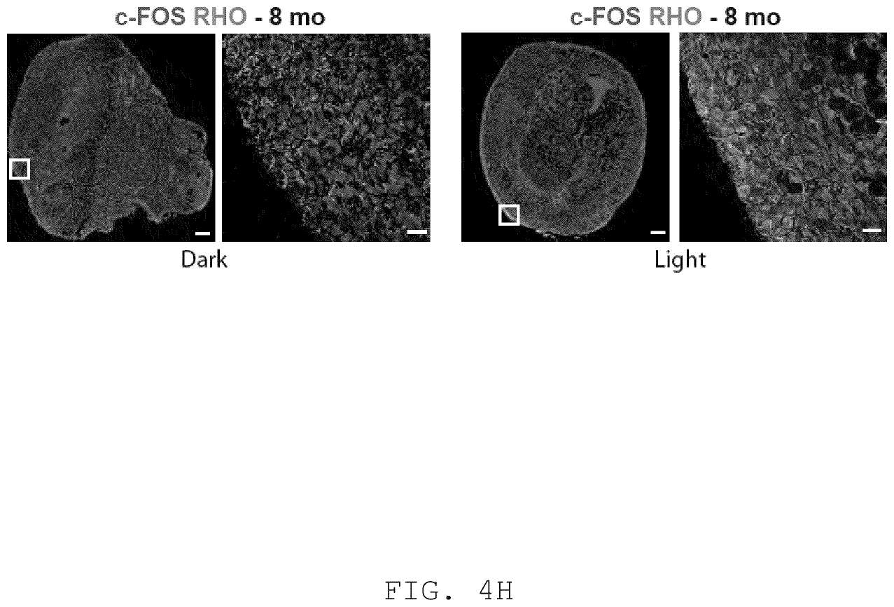

Human whole-brain organoids are largely self-patterning systems and therefore in principle have the potential to generate the vast cellular diversity of the endogenous developing human brain. However, this possibility remains largely untested. To address this point directly, we have developed a modified version of the culturing protocol first described by Lancaster et al.² to foster extended periods of growth and development in vitro and to favor the progressive generation of many cell types. Reducing the number of pluripotent stem cells used in initial EB seeding (2,500 cells), an enhanced neural induction paradigm, and addition of BDNF to the final differentiation medium have allowed us to successfully prolong the culture period, limiting premature cell death and enabling long-term, progressive development for 9-13 months (FIG. 1A, FIG. 5; see Methods for detailed protocol). With this protocol, the percentage of embryoid bodies that progressed to form long-term organoid cultures (>9 months) was greater than 95% when using the iPSC11a line and slightly lower (70%) using the HuES66 line.

To define the timeline of generation of broadly-defined cell classes, we first performed immunohistochemistry (IHH) for a small number of informative single-gene markers in organoids derived from the iPSC11a line. Specifically, we performed IHH analysis for markers of neural progenitors (Pax6), neurons (MAP2, GAB A, TH), and glial cells (GFAP, OLIG2) over a time course of organoid development spanning 1, 3, 6, and 9 months. We also examined the neural progenitor markers Nkx2.1, Gbx2, and Otx2 at 1 month. Similarly to the endogenous developing brain, 1 month organoids exhibited early brain regionalization, as shown by the expression of markers for germinal zones of the forebrain (Pax6, Nkx2.1), midbrain (Otx2), and hindbrain (Gbx2) (FIG. 5A). Pax6+ neural progenitors emerged first, followed by increased expression of the pan-neuronal marker (Map2) and markers for glutamatergic (Vglut1), GABAergic (GABA), and dopaminergic (TH) neuronal identities (FIG. 5B). Matching the endogenous developmental sequence, GFAP+ astroglia became apparent only at 3 mo, following the main wave of neurogenesis (FIG. 5B). Organoids also produced radially layered structures resembling the developing cerebral cortex. At 1 mo, PAX6+ progenitor cells surrounded the lumen of fluid-filled, ventricle-like structures (FIG. 5C), and at 6 mo, cells expressed class-specific cortical projection neuron markers, including markers of corticofugal and callosal projection neurons (FIG. 5C). The data indicate a developmental sequence of cell types and regional identities that broadly follows the temporal progression observed in vivo.

### Example 2—Large-Scale, Single-Cell Sequencing in Human Brain Organoids Reveals Extensive Diversity of Cells Resembling Known Endogenous Classes

Although single-marker analysis is valuable for gaining a global picture of the sequential generation of broad classes of cells and their geographic organization, individual markers cannot resolve the cellular diversity of the human brain, in which closely related sub-classes of cells can be identified only using combinatorial gene signatures. It also does not allow the identification of cell populations not expected a priori. Single-cell RNA sequencing allows systematic interrogation of many genes, but the unparalleled cellular diversity of the human brain requires that very large numbers of cells be profiled. To address this issue directly, we employed Drop-seq single-cell mRNA sequencing⁹ to molecularly profile 82,291 single cells isolated from 31 organoids derived from a healthy-control pluripotent stem cell line (iPSC line 11a; see Methods). Single cells from 3-month (15,402 cells) and 6-month (66,889 cells) organoids were sequenced to an average depth of ˜100,000 reads per cell. A matrix of read counts for each gene in each cell was assembled, and principal component analysis (PCA) was performed with dimensionality reduction using tSNE¹⁰⁻¹² (see Methods).

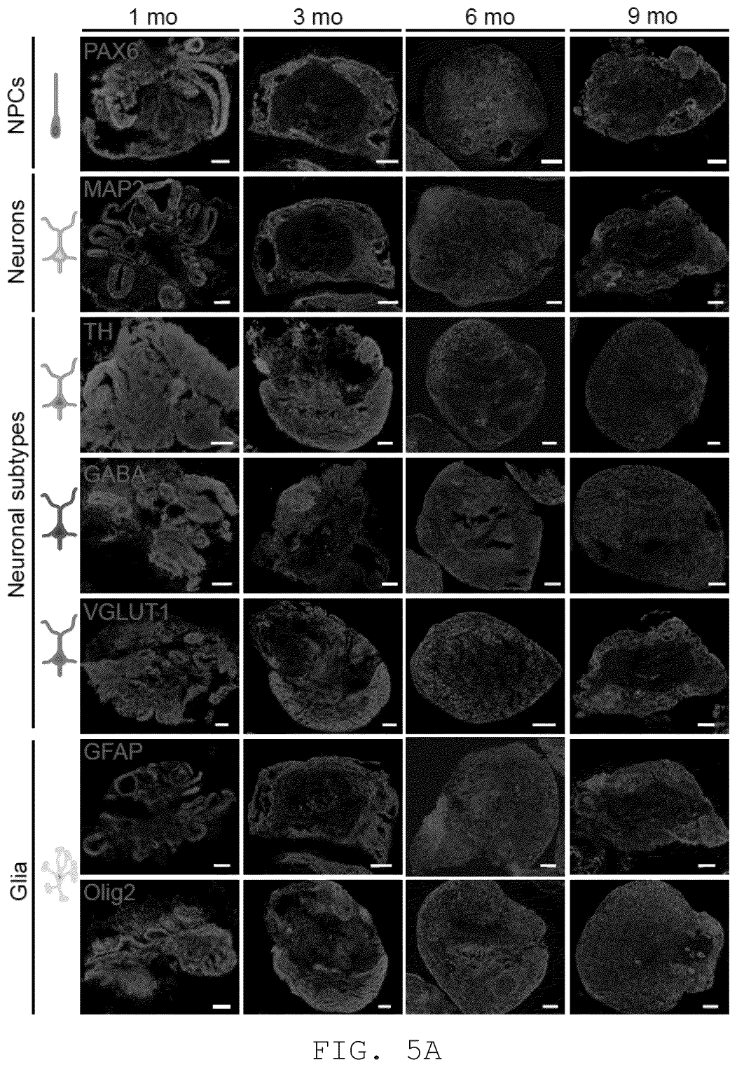

Clustering of all cells from the 6-month organoids based on their transcriptional profiles revealed ten main transcriptionally distinct groups of cells (FIG. 1B). In order to decode the identity of cells present within each cluster, we systematically compared the signatures of differentially expressed genes within clusters or their sub-clusters (see below) to gene signatures extracted from existing RNA-seq datasets of distinct endogenous cell types (for sources see FIG. 6B). This approach identified cell populations based on the combinatorial expression of many genes, rather than single markers. We were able to define the identity of the seven largest clusters (c1, c2, c3, c4, c5, c9 and c10) (FIG. 1B). Although it is important to take into account possible under-sampling issues due to different cell viability upon dissociation, we were able to molecularly identify many different classes of cells, including forebrain excitatory neurons, which are notoriously fragile¹³.

Six of the seven clusters that we could define belonged to the neurectodermal lineage; however, one cluster (c1) expressed mesodermal markers, including several muscle-specific genes (such as Myogenin) and several Myosin genes (MYH3, MYH8, MYL1, MYLPF), indicating that, despite early patterning to a neurectodermal fate, organoids can still produce a minority of cells of another embryonic origin.

Cluster c2 (8,409 cells) was primarily composed of cells expressing genes identified in endogenous adult human astrocytes¹⁴, including canonical astrocyte markers AQP4 and GFAP. Cluster c9 (17,103 cells) contained progenitors of neuroepithelial origin, including cells with oligodendrocyte precursor-like identity, as suggested by the expression of marker genes previously found in human oligodendrocytes and their progenitors^{15,16}. Cluster c10 (13,428 cells) was mainly composed by cells with signatures of highly proliferative progenitors (e.g., TOP2A and MKI67). Cluster c3 (971 cells) was enriched for cells expressing neuronal genes, including dopaminergic markers (e.g., TH and EBF1).

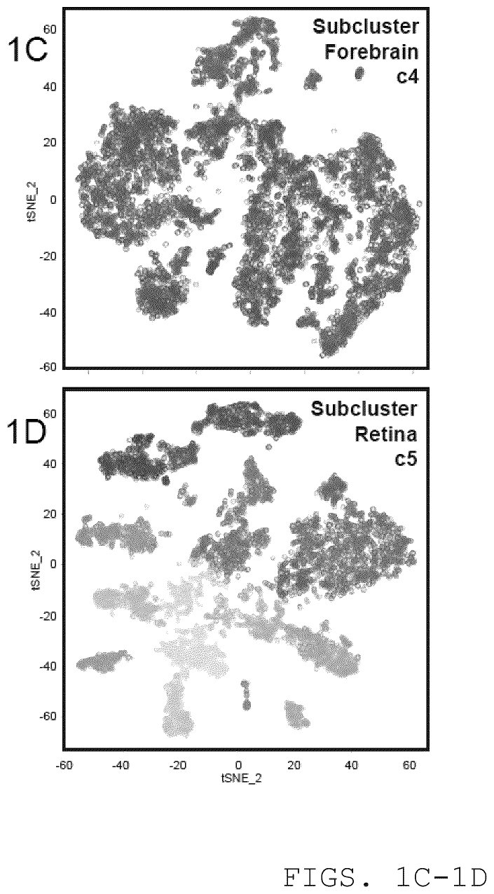

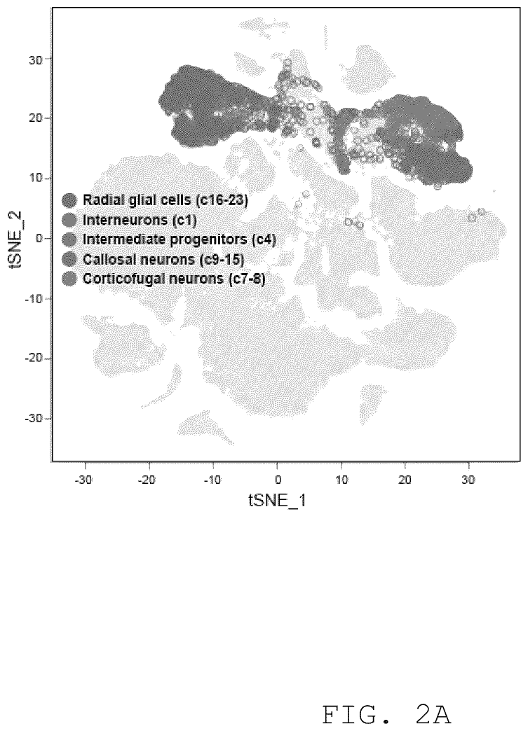

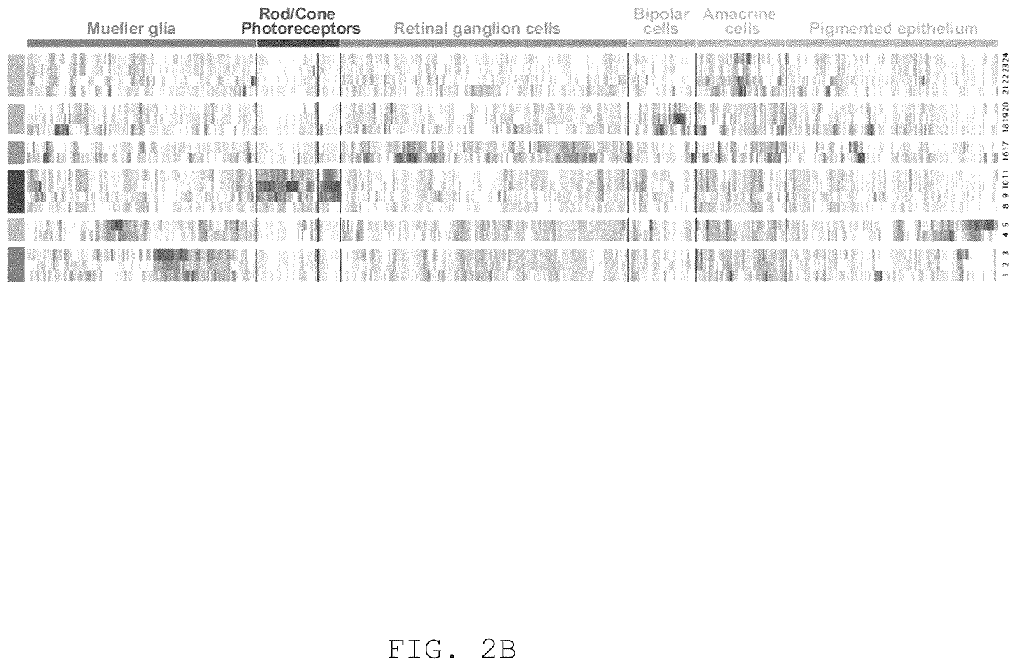

Perhaps the most interesting clusters were c4 and c5 (12,378 and 9,919 cells, respectively) (FIGS. 1B-1D). Cluster c4 had a clear neuronal signature, expressing genes such as MAP2 and GAP43. Further examination revealed enrichment for markers of neurons and radial glia cells of the cerebral cortex. To better ascertain the diversity within this group of cells, we performed a second iteration of clustering analysis on this specific cell population. This sub-clustering analysis identified 30 transcriptionally distinct sub-clusters of cells within cluster c4 (FIG. 1C); we were able to assign 17 of these to 5 broad cell type identities (FIG. 2A). We observed a striking separation of sub-clusters 7-8 from sub-clusters 9-15, which included neurons of putative corticofugal and callosal projection neuron identity, respectively. Using a published single-cell RNAseq profile of human cortical radial glial cells¹⁷ as a reference, we observed enriched expression of human radial glial markers in sub-clusters 16-23, while sub-cluster 4 was distinguished by expression of canonical genes for intermediate progenitors, including EOMES and ELAV4. Finally, sub-cluster c1 displayed molecular signatures of interneurons, including GAD1 and GAD2. We conclude that cluster c4 contains distinct cell types of the forebrain, including putative cortical excitatory and inhibitory neurons and different cortical progenitor cells.

Cluster c5 (9,919 cells) showed a strong representation of genes of the neural retina, such as the photoreceptor markers CRX and RCVRN^{18,19}. Comparison with several gene expression datasets of mouse and human retina^{9,20,21} confirmed this cluster was composed predominantly of retinal cells. The availability of a previously published Drop-seq analysis of 44,408 single cells from the murine retina 9 allowed us to very finely decode the cellular composition of cluster c5 by comparing across all of the genes identified as class-specific retina markers by Macosko et al. 9.

We found that cluster c5 contained virtually all of the cell classes found in the mouse retina. Specifically, analysis of each sub-cluster showed the presence of Muller glia cells (e.g., DKK3, SOX9), canonical pigmented epithelial cells (e.g., MLANA, MITF), photoreceptors (e.g., CRX, RCVN, NRL), retinal ganglion cells (e.g., NEFL, NEFM), bipolar cells (e.g., LHX4, PCP2) and amacrine cells (e.g., TFAP2B, SLC32A1) (FIGS. 2B-2D).

Together these data demonstrate that whole-brain organoids have the potential to generate unprecedented cellular diversity in vitro, including the development of multiple neuronal lineages from distinct regions of the brain and the eye. This degree of cell-type complexity has never been reported in any in vitro differentiation system.

### Example 3—Protracted Culture of Human Brain Organoids Leads to Increased Cell Diversity and Advanced Neuronal Maturation

In order to understand whether there is a progressive generation of cell types and possibly distinct maturation states over time in culture, we performed Drop-seq analysis on an additional 15,402 single cells from organoids at 3 months (n=12 organoids from 2 flasks) derived from the same pluripotent stem cell line (iPSC11a). We found that while some clusters of cells were present at both 3 and 6 months, older organoids contained additional clusters not present at 3 months (data not shown). This indicates that over the additional 3 months in culture, organoids not only survived but also continued to develop, generating new cell types.

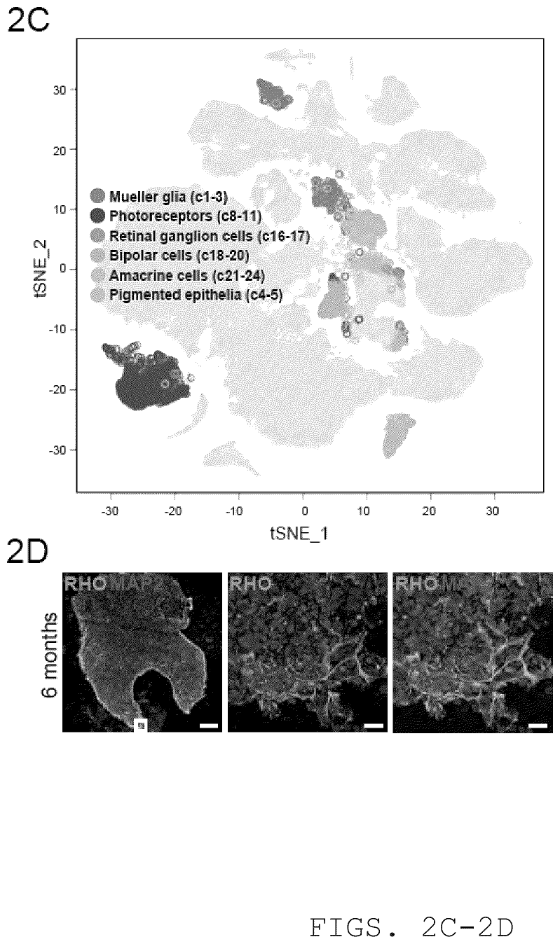

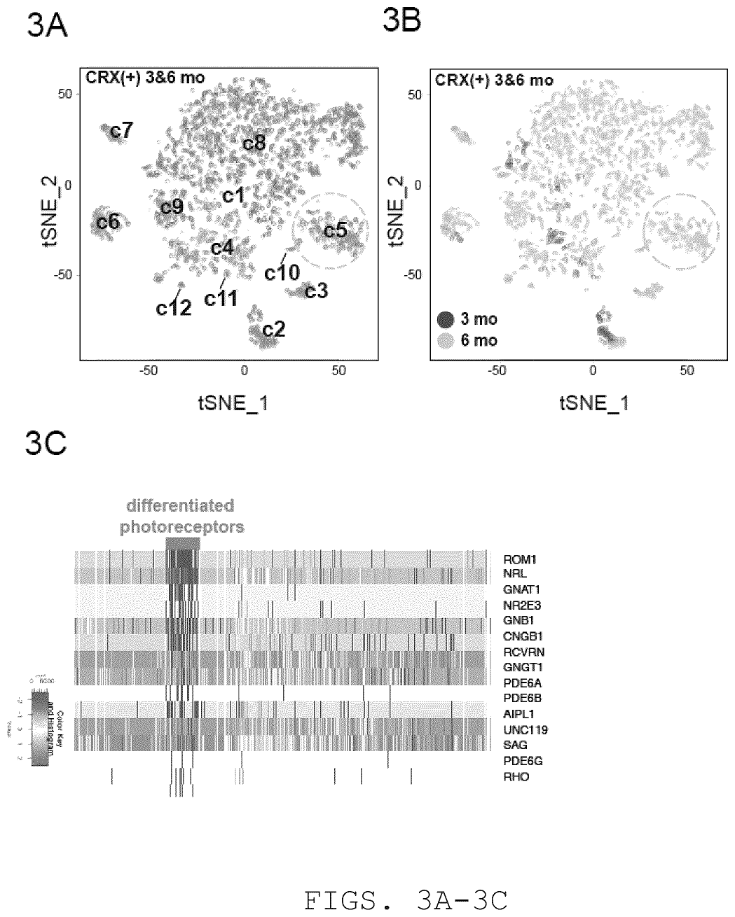

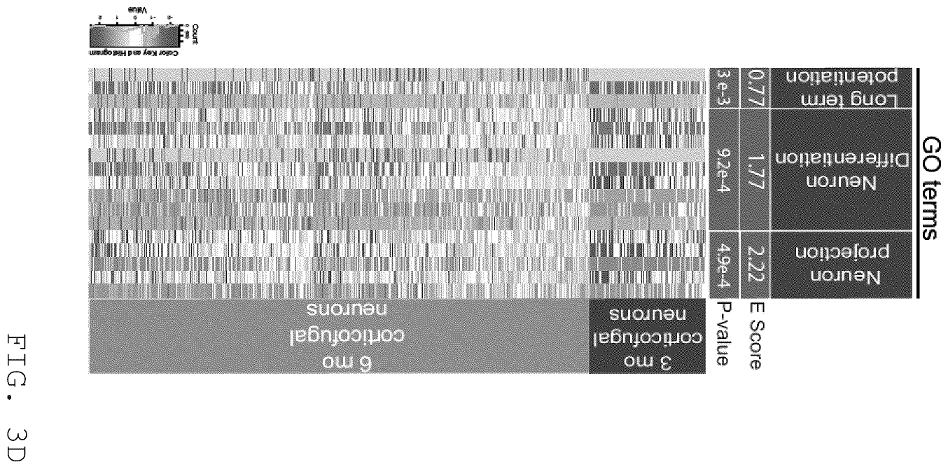

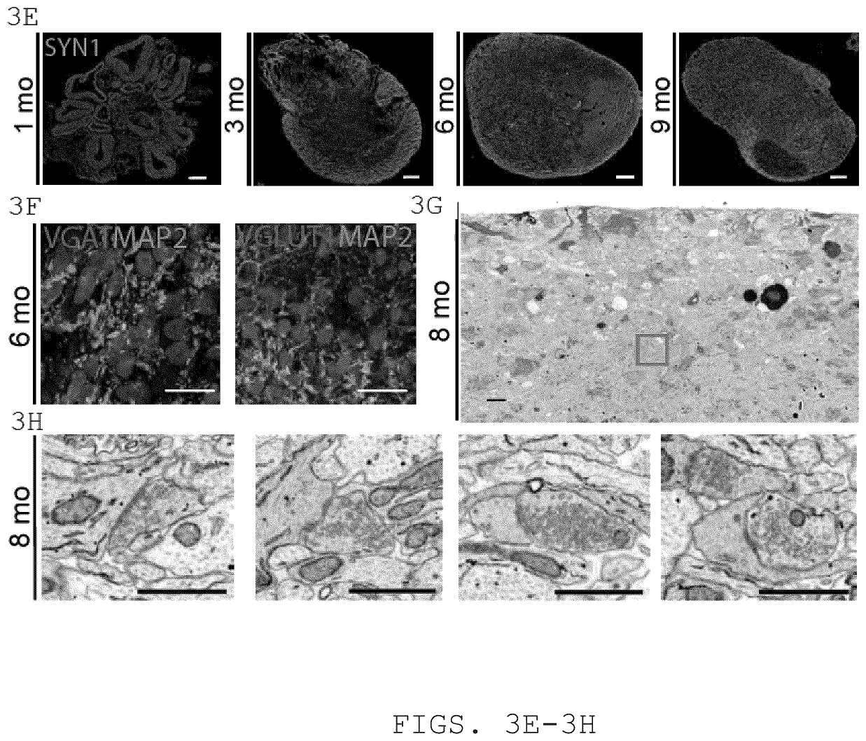

To investigate whether long-term culture also allows greater cell maturation, we identified populations of cells that were present at both 3 and 6 months and compared their molecular signatures between ages. First, we examined cells in the retina cluster that expressed the transcription factor CRX, a known marker of both developing and mature photoreceptors (developing and mature photoreceptors can be easily differentiated at the molecular level). Clustering analysis of all CRX-positive cells across both 3 and 6 month organoids revealed 12 transcriptionally defined clusters (FIGS. 3A-3B). Among the cells present only at 6 months, we concentrated on cluster c5 (168 cells) as one cluster that did not contain any 3 month cells even when controlling for potential under sampling at 3 month (chi-square analysis, p-value: 1.6×10⁻⁴).

These cells showed differential expression of key genes involved in phototransduction, including the alpha subunit of rod transducin (GNAT1), the alpha and beta subunits of the rod cGMP-phosphodiesterase (PDE6A, PDE6B), and the rod outer membrane protein (ROM1) (FIG. 3C); all genes expressed in photoreceptors at an advanced state of differentiation. Accordingly, a Gene Ontology (GO) term search performed on genes enriched in cluster c5 showed significant enrichment in genes involved in phototransduction and the rhodopsin-mediated signaling pathway (data not shown). We conclude that photoreceptors progressively matured within organoids between 3 and 6 months and that long-term cultures of human brain organoids fostered the differentiation of photoreceptors equipped with key proteins involved in light responsiveness.

Cells of putative corticofugal projection neuron identity, like photoreceptor cells, were present in both 3 and 6 month organoids. Confirming progressive maturation, functional clustering of gene sets (using DAVID 6.7²²) across genes differentially expressed in these neurons at 3 month versus 6 month revealed a clear enrichment at 6 month for genes involved in neuronal maturation (FIG. 3D).

We then investigated whether the progressive maturation of cells detected by molecular profiling was also reflected in the generation of structural traits typical of more mature neurons, including formation of synapses and the presence of dendritic spines.

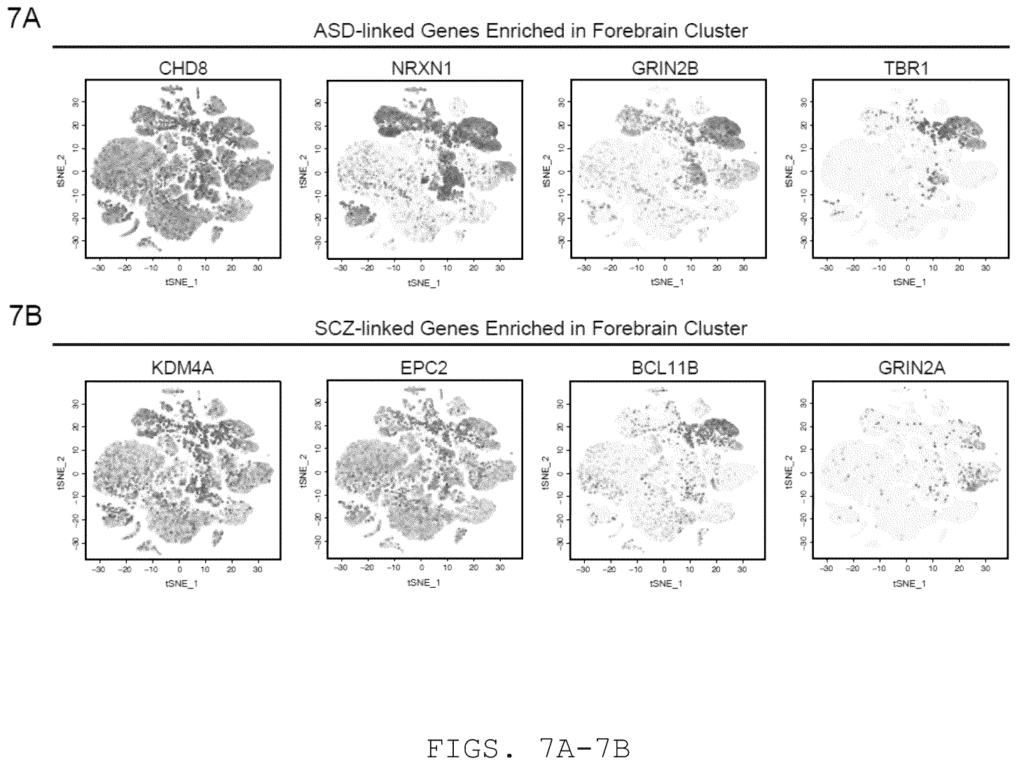

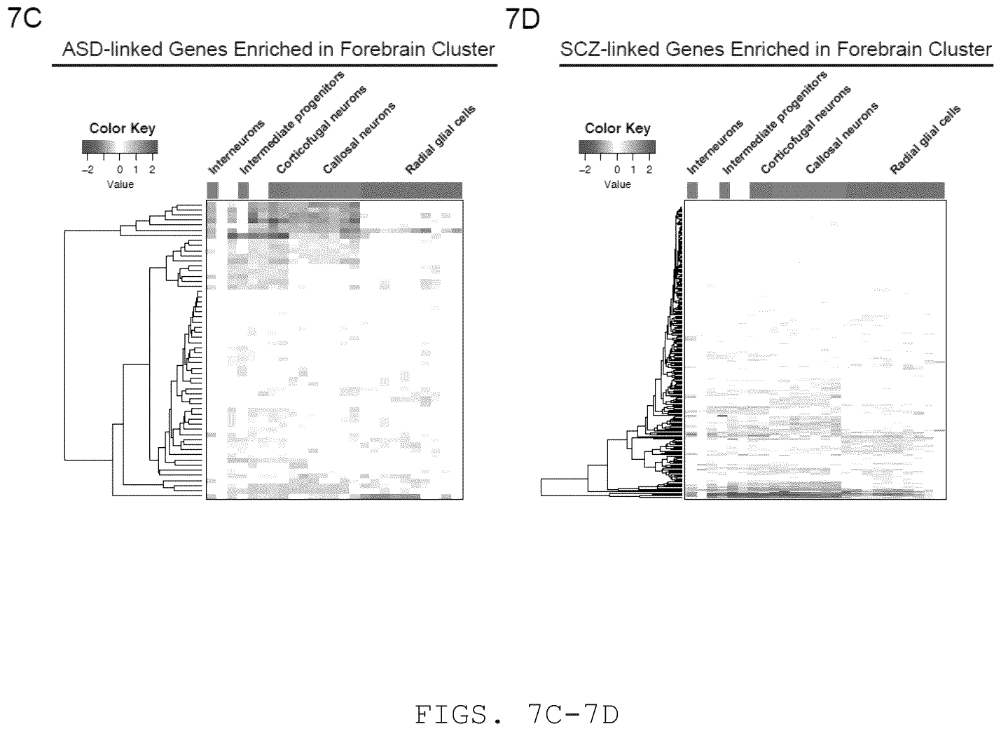

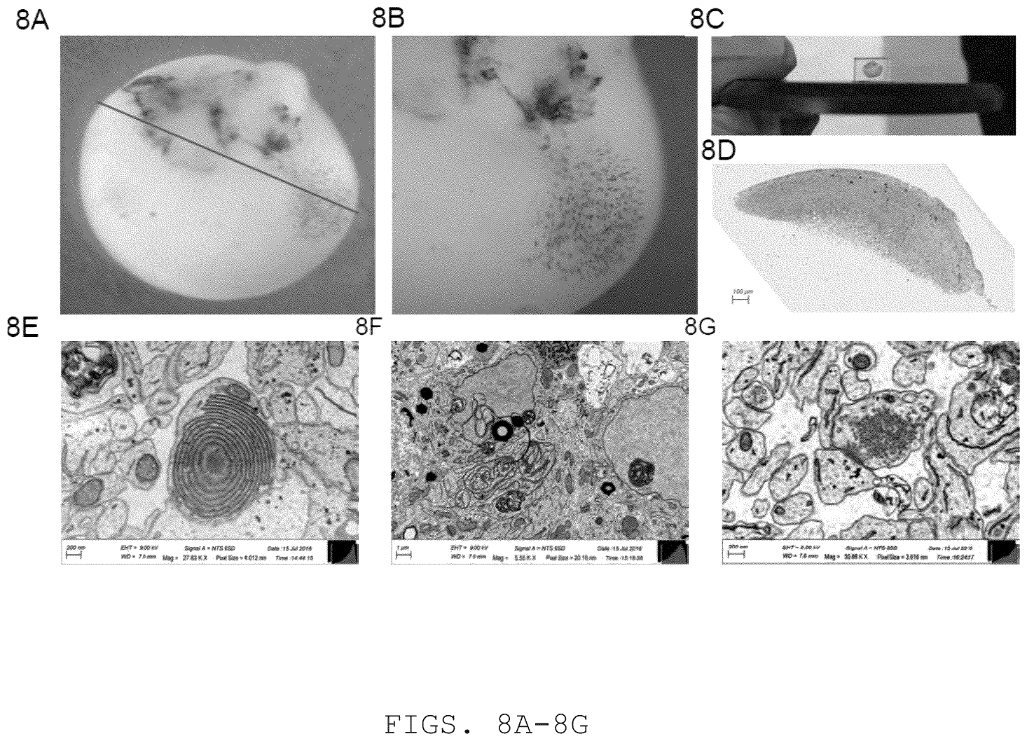

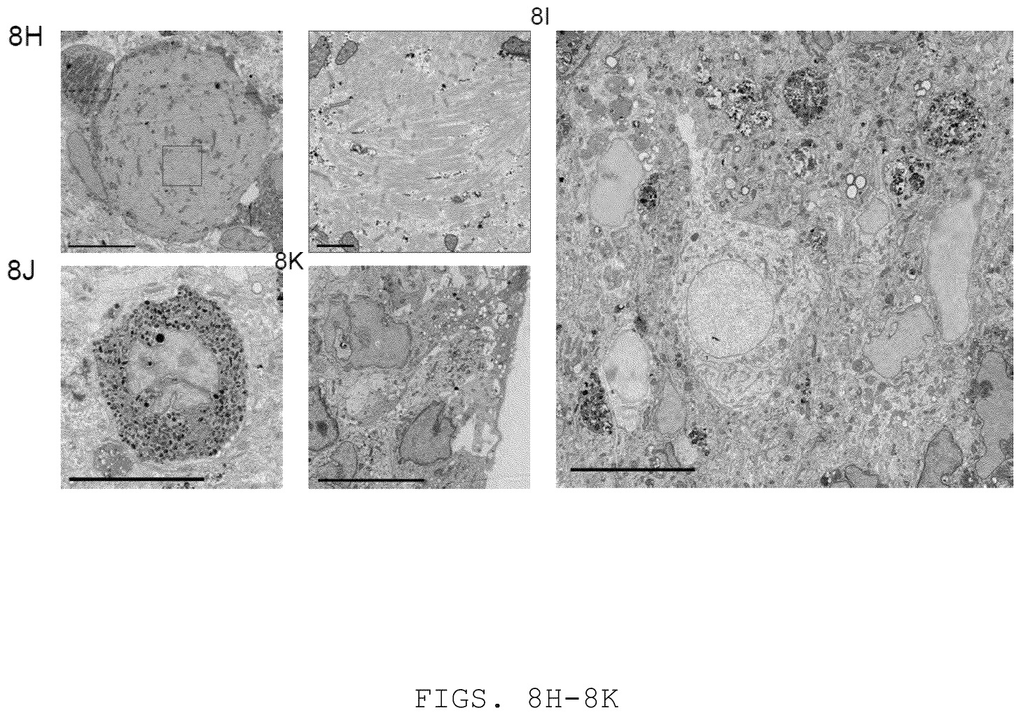

First, we followed the expression of synaptic markers over time in culture. The pre-synaptic marker synapsin 1 (SYN1) was absent at 1 month but began to be expressed at 3 mo, persisting for at least 9 month in culture (FIG. 3E and data not shown). Notably, initial expression of SYN1 coincided with the development of astroglia (FIG. 5B), which in vivo play critical roles in synaptogenesis and synaptic maturation²³. Detection of VGAT+ and VGLUT1+ puncta suggested that both glutamatergic and GABAergic synapses were present (FIG. 3F). In order to confirm these findings at the structural level, we performed Electron Microscopy (EM) imaging of an organoid at 8 months. The organoid was sectioned at 40 nm slice thickness, collected on tape, and imaged on a scanning electron microscope²⁴. We could detect a wide range of cellular morphologies (FIG. 8) and many structurally-defined synapses (FIG. 3H).

Next, we concentrated on neuronal morphology and in particular on dendritic spines, which are notoriously difficult to generate in vitro due to the fact that neurons derived from pluripotent stem cells typically retain an embryonic identity. We chose a region about 70 μm below the surface of an 8 month organoid for serial reconstruction (FIG. 3G). We collected a stack of 136 consecutive slice images (1 slice lost), generating a volumetric data set of approximately 16×16×5.4 μm. We identified a total of 129 synapses in this volume (0.088 synapses per μm³) (FIG. 3H). We analyzed 100 of these synapses and identified both pre- and postsynaptic processes. We found that with two exceptions (two objects both receiving and making synapses), those processes were either exclusively pre-synaptic (57 objects, axons) or exclusively post-synaptic (54 objects, dendrites), often making more than one synapse in the volume (FIG. 3I). Axons, more than dendrites, appeared to preferentially run parallel to the organoid surface (Wilcoxon rank-sum test, p<10⁻⁵, FIG. 3I). The labeled dendrites had a total of 37 spines, which were variable in shape, but were often innervated by a synapse. 30 of the 100 synapses were made on spines (FIGS. 3J-3K).

Together, these data indicate that human brain organoids continue to develop and mature over months in culture, generating increased cellular diversity and unprecedented neuronal maturity.

### Example 4—ASD and Schizophrenia-Associated Risk Genes are Expressed Across Distinct Cell Types in Human Brain Organoids

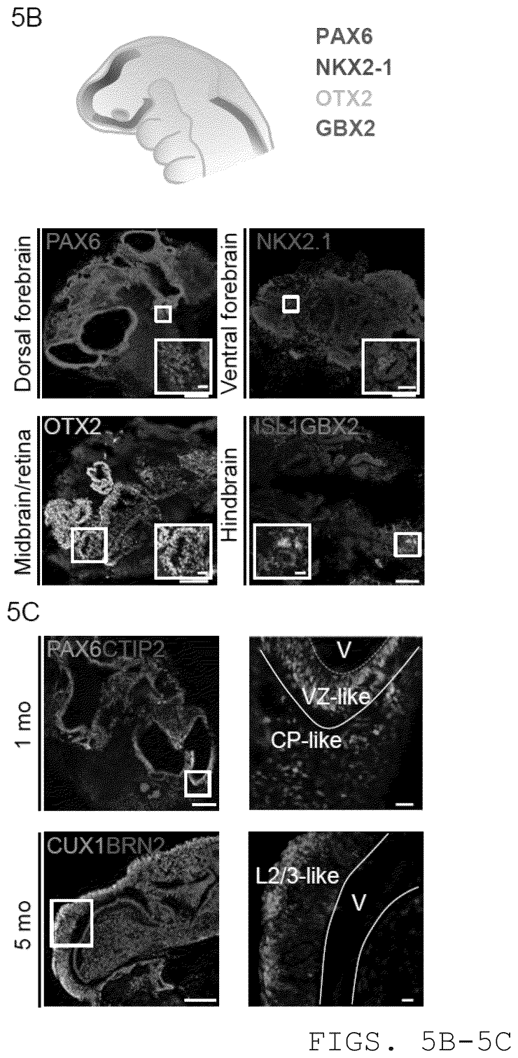

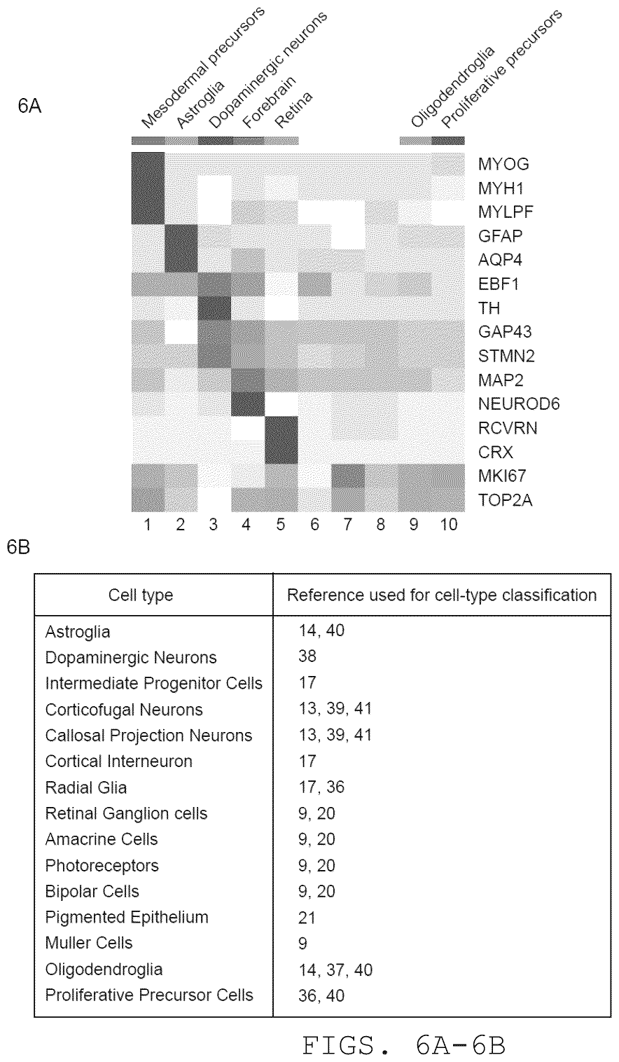

A wealth of genetic information on neuropsychiatric diseases has recently become available. To understand whether organoids may be good models to probe the effect of genetic mutations on human brain development and function with cell type-specificity, we tested whether expression of disease-linked genes^{25,26} mapped preferentially to any of the 10 main cell clusters identified at 6 months (c1-c10 in FIG. 1). Though disease-linked genes displayed distinct profiles of expression across multiple cell clusters (FIGS. 7A-7B), the largest proportion of disease-linked genes mapped to cells of the forebrain cluster (c4) (FIGS. 7A-7B, and data not shown), which represents cells of the cerebral cortex, a brain region whose function is compromised in ASD and SCZ. To increase the resolution of analysis within c4, we further examined expression within each of its sub-clusters (FIG. 7C). At this finer level of analysis, we found that compared to radial glia progenitors, neurons showed differential expression of a larger number of risk genes (for both disorders). In addition, individual genes showed enrichment in distinct subclusters within c4, suggesting cell-type-specific patterns of expression. These results point at cell-type specific enrichment of risk genes in distinct cell populations generated within organoids, suggesting that organoids will be useful for modeling the effects of disease-implicated variants on the development and function of distinct cell types.

### Example 5—Long-Term Cultures of Human Brain Organoids Allow Development of Spontaneously Active Neuronal Networks Responsive to Physiological Sensory Stimuli

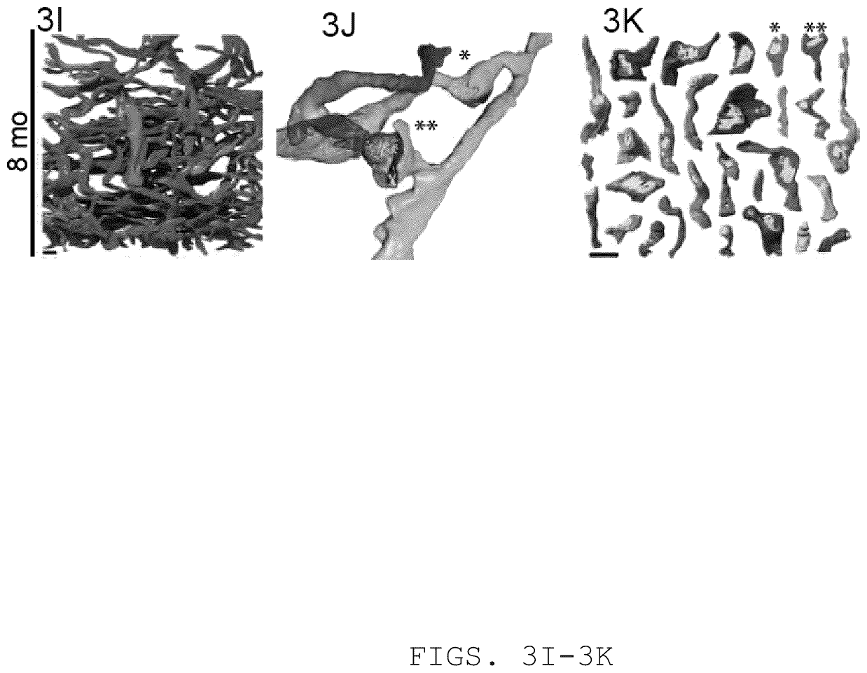

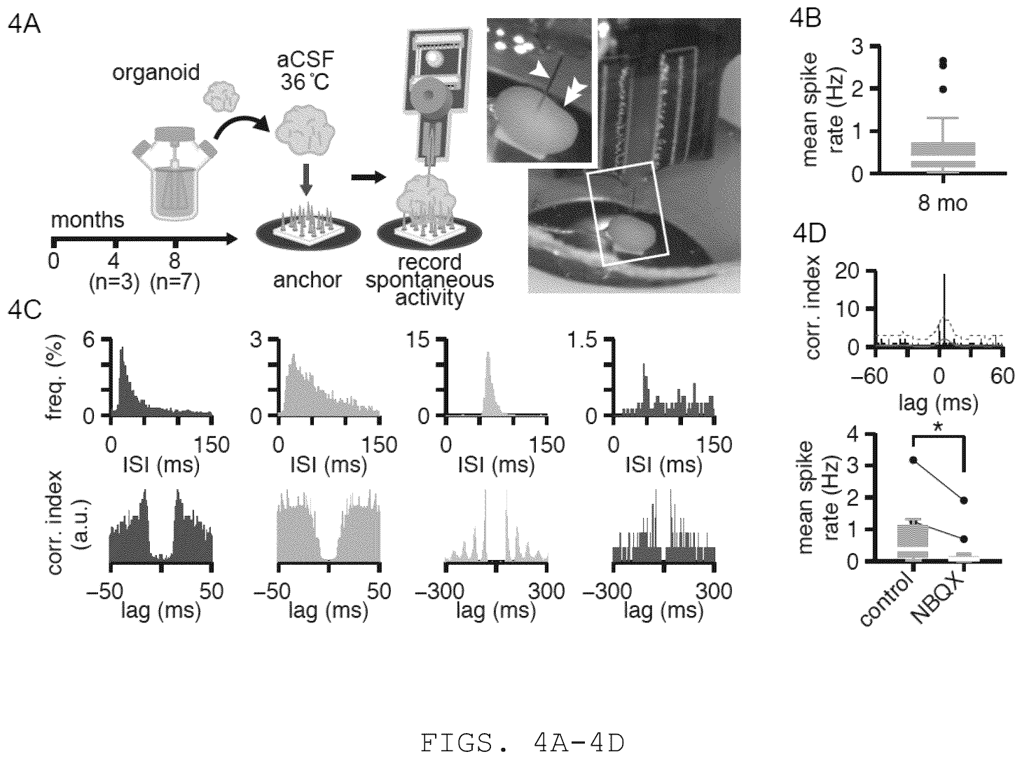

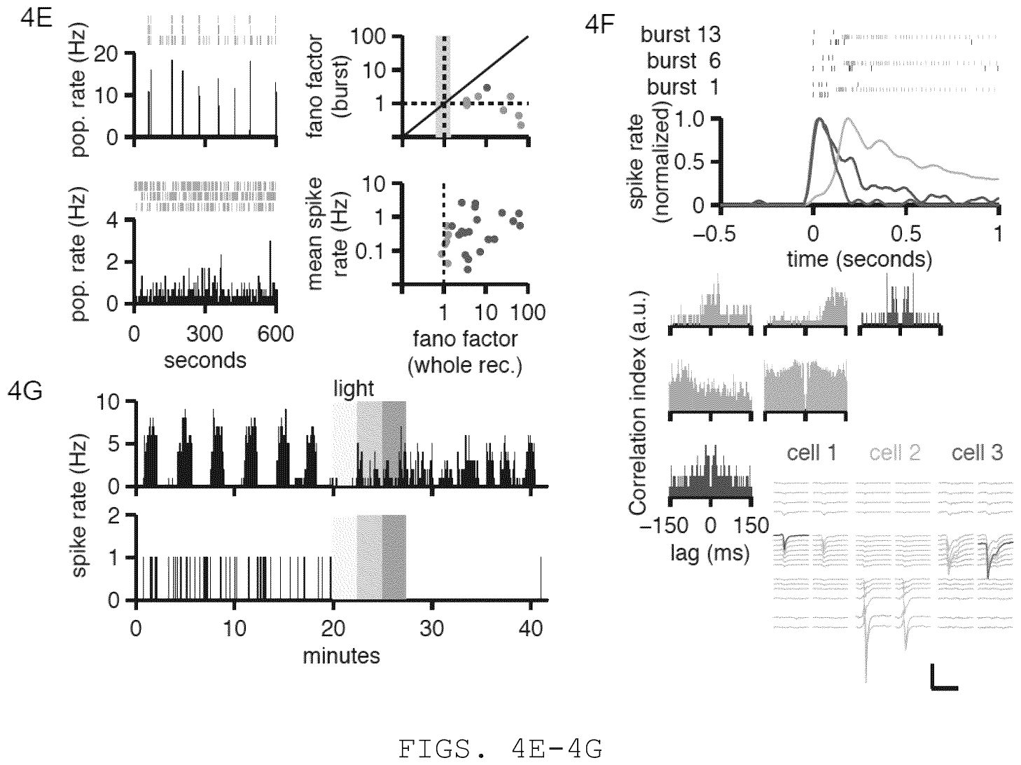

Given the enhanced maturity of the neurons generated in long-term organoid cultures, we investigated whether spontaneously active neurons and neuronal networks were present in 4 and 8 month organoids (FIG. 4A). To facilitate single unit isolation, we performed extracellular multiunit recordings with high-density silicon microelectrodes (Scholvin et al., 2016) (n=3 organoids at 4 mo; n=7 organoids at 8 mo; all recordings were performed in the absence of ambient light; FIG. 4A). We were able to isolate spikes (action potentials) in six out of seven 8 month organoids (11 recording sites and 31 neurons/units in total). In all cases tested, spike rate was suppressed by the bath application of TTX (2 μM), a voltage gated sodium channel antagonist (data not shown), confirming that spikes are action potentials. The mean firing rate of the recorded units was 0.36 Hz (95% CI=0.22 to 0.60, n=31 units from 6 organoids; FIG. 4B). Closer examination of the time-series data highlights the presence of distinct firing patterns in the neurons sampled (prototypical examples are shown in FIG. 4C), in agreement with the finding that molecularly different types of neurons are present within organoid.

We observed isolated positive peaks with a short lag time (<5 ms) in cross correlograms, indicative of excitatory mono-synaptic connections (FIG. 4D). Therefore, to determine if synaptic activity was driving the observed spike trains, we blocked AMPA-receptor mediated synaptic transmission with the bath application of 20 μM NBQX. The application of NBQX significantly reduced the median mean firing rate by 81% (FIG. 4D; baseline median firing rate=0.3800 Hz, 95% CI 0.1095 to 1.2072; NBQX-treated median firing rate=0.0733 Hz, 95% CI 0.0023 to 0.2048; p=0.0005, n=12 units from 4 organoids), indicating that the majority of recorded spikes are being driven by fast non-NMDA-receptor mediated excitatory synaptic transmission.

We then investigated whether spontaneously-active neuronal networks developed within long-term cultures of organoids. We recorded population firing rates in 6 organoids at 8 months. We found that in 3 out of 6 organoids, population firing rates were relatively stable (6 out of 6 recording sites, FIG. 4E bottom left panel). However, in the remaining 3 organoids, 5 out of 5 recording sites showed obvious non-stationary firing patterns (FIG. 4E, top left panel), displaying periods of increased activity (order of seconds) separated by longer periods of relative quiescence (lasting 10's of seconds). Notably, in 6 out of 6 organoids, the time-series fano-factor for the majority of all recorded units was greater than 1, indicating that single unit firing is not well modeled by a stationary Poisson distribution, and pointing at the presence of network activity (FIG. 4E, bottom right).

To understand whether neurons were stably recruited into the networks, we characterized the firing patterns of individual neurons during upstates. Time series data was aligned to upstate onset (4 recording sites from 2 organoids). During the first second of the upstate, the median of the neurons' mean firing rate was 13.2 Hz (9 cells, 4 recording sites, 2 organoids) and the fano-factor was close to 1, consistent with a stationary Poisson distributed system and implying a fixed firing profile (FIG. 4E, top right panel). These data indicate that once recruited into an active network individual neurons behave reproducibly.

We next sought to determine whether neurons in 8 month organoids showed coordinated firing activity. We examined recordings containing multiple isolated units and identified sets of neurons displaying periods of coordinated activity (3 recording sites from 2 organoids displaying non-stationary firing). Notably, within these periods the order of neuronal recruitment and firing showed a clear temporal structure (FIG. 4F, top and middle panel). In contrast to 8 month organoids, no active units were sampled in 4 month organoids (3 out of 3 organoids), indicating that brain organoids take several months to develop neural networks that are sufficiently mature to display spontaneous, coordinated activity. Together, these results indicate that whole-brain organoids are intrinsically capable of establishing non-stationary neuronal networks that can support self-organized patterns of activity.

To date, it has not been possible to use physiological stimuli to modulate the activity of human neurons and neuronal networks differentiated in vitro unless optogenetic approaches are used. Our single-cell RNAseq analysis indicates that long-term cultures of human brain organoids support the differentiation and maturation of photoreceptor-like cells equipped with key proteins for light responsiveness. We therefore decided to test whether light-based, physiological sensory stimuli could activate light-sensing cells and consequently modulate downstream neuronal activity within organoids. We selected one organoid that exhibited intermittent periods of pronounced activity and stimulated it with 530 nm light (90 pulses of 200 ms duration were delivered at 0.2 Hz: 30 pulses at 30 μW/cm², 30 at 100 μW/cm², and the final 30 at 300 μW/cm²). We found that in two isolated units light application specifically disrupted ongoing firing patterns (FIG. 4G). In agreement, cFos, an activity-dependent gene, was upregulated in 8mo organoids that were exposed to light compared to control organoids kept in the dark, further supporting the finding that light stimulation is capable of modulating the activity of light-photosensitive cells that develop within this organoids.

These results indicate that long-term cultures of human brain organoids can develop spontaneously active neurons and neuronal networks. This capacity matures over time, in line with our molecular observations of progressive neuronal maturation. In addition, the development of photoreceptor-like cells within organoids provides a unique platform to modulate and study neuronal activity in response to physiological stimuli.

### DISCUSSION

Human brain organoids have enormous potential to serve as in vitro models of the human developing brain. Fulfillment of this promise however requires a deeper knowledge of the cellular composition of organoids and an understanding of the extent to which they can generate the extensive cellular diversity of the brain and also produce mature neuronal traits and functional neuronal networks. Here we report that 3-dimensional human whole-brain organoids can achieve extensive in vitro development and maturation and generate an unprecedented diversity of cell types, which we could map through the largest single-cell transcriptional profile realized to date. Complex lineage-fate decisions are executed in culture to ultimately form many cell classes from both the brain and the eye, including all known cell types of the retina and diverse classes of neurons and progenitors of the cerebral cortex. While in the future it will be important to determine whether most organoids derived with this protocol have comparable capacities to generate this heterogeneity of cell types, the data demonstrate the potential that organoids have to self-govern the differentiation of a large spectrum of the cells found in vivo. At a more fundamental level, these results also highlight the outstanding capacity that the brain has to intrinsically control the development of its cellular constituents down to very fine cell-lineage decisions.

Directed differentiation of CNS neurons from pluripotent stem cells typically results in the production of cells with predominantly embryonic traits. We find that the cellular composition of organoids diversifies over time in culture and displays progressive levels of maturity. This is notably reflected in the acquisition of structural traits characteristic of mature neurons, including dendritic spine-like structures, which have been notoriously difficult to generate by directed differentiation in culture. This offers the opportunity to study a new set of developmental processes, such as human synaptic pruning and active spine refinement events, which are intimately linked, for example, to neuropsychiatric diseases, but could never before be modeled in the dish.

In agreement with an advanced state of maturation, whole brain organoids progressively generate, between 4 and 8 months in culture, spontaneously-active neurons and neuronal networks. During the same period of time, photoreceptor-like cells mature substantially and become responsive to non-invasive, light-based sensory stimulation that appears capable of affecting neuronal activity. This suggests that co-development of brain and retina cells within the same organoid may in the future serve as a new experimental system to investigate the fine response of neuronal networks to physiological sensory stimuli, rather than using engineered optogenetic systems.

The robustness of the neuronal networks, the presence of structural traits of mature neurons, and the opportunity to use sensory experience to finely modulate neuronal activity collectively suggest that, beyond modeling early events of progenitor biology, 3D brain organoids have the potential to model higher-order functions of the human brain that rely on circuit functionality and plasticity. This is of importance for modeling the complexity of neurodevelopmental and neuropsychiatric pathologies where circuit functionality is disordered. In this regard, our finding that risk-genes for schizophrenia and ASD are expressed with cell type-specificity in human brain organoids further highlights the value of these models for investigate the role of genetic variants in these complex pathologies.

The present invention includes a culturing protocol that supports prolonged development of human whole-brain organoids for more than 9 months. Using high-throughput single cell mRNA sequencing (Drop-seq), more than 80,000 individual cells were analyzed, providing the largest-to-date molecular map of the diversity of cell types in any organ system. It is demonstrated that the immediate utility of Drop-seq is to detect cell-type specific expression patterns of genes linked to mental illnesses in organoids by uncovering associations between risk genes and cell types. It is shown that organoids undergo protracted development in vitro allowing for substantial neuronal maturation, including generation of dendritic spines and the formation of spontaneously active neuronal networks. Finally, an initial demonstration is provided showing that the activity of neurons within organoids can be modulated using light-based stimulation of spontaneously-developing light-sensitive cells, indicating that organoid models may in the future be used for investigation of circuit functionality in response to physiological sensory stimuli.

### Methods

### Pluripotent Stem Cell Culture

Human pluripotent stem cell lines used in these experiments were hiPSC11a²⁷ and HUES66²⁸. Cells were maintained in 10-cm tissue culture dishes (Corning) coated with 1% Geltrex membrane matrix (Thermo Fisher Scientific) in mTeSR medium (Stemcell Technologies). PSCs and colonies were dissociated with Gentle Cell Dissociation Reagent (Stemcell Technologies). All human pluripotent stem cells were maintained below passage 50 and confirmed negative for mycoplasma.

### Long-Term Cultures of Human Brain Organoids

Cerebral organoids were generated using a modified version of the protocol published by Lancaster et al.²⁹ (Nature Protocols, 9.10:2329 (2014). Embryoid bodies (EBs) were derived by dissociating PSCs colonies and plating 2,500 single cells in each well of a 96-well plate. EBs were cultured as described by Lancaster.²⁹ After five days in culture, EBs were transferred to Intermediate Induction Medium (IIM), consisting of DMEM/F12, 6% KOSR (Thermo fisher scientific), 0.9% FBS (GIBCO), 6 ng/ml bFGF (Peprotech), 0.7% N2 supplement (Invitrogen), Glutamax (Invitrogen), minimum essential-media-non essential amino acid (MEM-NEAA) (Thermo fisher scientific), heparin 0.7 μg/ml (Sigma). Two days after plating in IIM, 500 μl of Neural Induction Medium (NIM) were added to each well. Following neural induction, organoids were embedded in Matrigel and transferred to cerebral differentiation medium (CDM) as described by Lancaster et al. A total of 40 organoids were added to each spinner flask, and medium was changed once every six days for the duration of the culture process. After one month, brain derived neurotrophic factor (BDNF) was supplemented to the CDM medium at a concentration of 14 ng/ml. Cerebral organoids were cultured for up to 13 months.

### Histology and Immunofluorescence

Cerebral organoids were fixed by immersion in 4% paraformaldehyde (Electron Microscopy Services), cryoprotected in 30% sucrose solution and then embedded in optimum cutting temperature (OCT) compound (Tissue Tek). Organoids were then cryosectioned at a thickness of 14 or 30 μm.

Immunohistochemistry was performed as previously described³⁰. Primary antibodies and dilutions used were as specified in Table 1. Goat anti-rabbit IgG Alexa Fluor 488, 546 and 647 (Life Technologies A11070, A-11071, A21246), goat anti-chicken IgG Alexa Fluor 488, 546, 647 (Life Technologies A11039, A11040, A21449), goat anti-rat 488, 546, 647 (Life Technologies A11006, A11081, A21247), goat anti-mouse IgG Alexa Fluor 488, 546, 647 (Life Technologies A11017, A11018, A21237), Alexa Fluor donkey anti-mouse 488 (Life Technologies 715-545-151), Alexa Fluor donkey anti-rabbit 488 (Life Technologies A10036) Alexa Fluor donkey anti-rat 488 (Life Technologies A-21208), Alexa Fluor donkey anti-mouse 546 (Life Technologies A10036), Alexa Fluor donkey anti-goat 546 (Life Technologies A-11056), Alexa Fluor donkey anti-mouse; A31571, Alexa Fluor donkey anti-rabbit 647; A10040 secondary antibodies were diluted 1:1200. Rhodopsin immunohistochemistry was performed without Triton X-100 or Tween 20 to preserve membrane integrity, and signal was amplified using biotin (1:500, Vector BA9200) and streptavidin (1:600, Thermo Fisher 532354) conjugates. Cell nuclei were stained with Hoechst staining (1:5,000; Invitrogen), and slides were mounted using Fluoromount-G mounting medium.

### Microscopy and Image Analysis

All mounted sections were imaged using a Nikon Eclipse Ti microscope, analyzed with NIS Elements analysis software and processed using ImageJ. Confocal images were obtained using a Zeiss 700 confocal microscope. Images were collected and analyzed using the Zen 2010 program. For EM, images were processed using ImageJ, manually segmented using VAST Lite (freely available on the World Wide Web at subdomain software.rc.fas.harvard.edu/lichtman/vast/), and rendered in 3ds Max (Autodesk, Inc).

### Drop-Seq Single Cell Sequencing

Droplets containing single cells and barcoded micro-particles were generated and processed as described in Macosko⁹. Briefly, droplets were collected and beads were recovered and processed for immediate reverse transcription. The resulting cDNA was amplified, fragmented and further amplified using the Nextera XT DNA library preparation kit. Sequencing was performed on the Illumina NextSeq 500.

Clustering of cells derived from 6-month organoids was performed using the Seurat R package¹⁰, with some modifications from the procedure described previously (Macosko et al., Cell, 2015). Clustering was done in two iterative rounds of principal components analysis (PCA). First, digital gene expression matrices were column-normalized and log-transformed. Cells with fewer than 400 expressed genes were removed from analysis. A set of variable genes was then identified by binning the average expression of all genes into 300 evenly sized groups and computing the median dispersion (variance divided by the mean) in each bin. Genes were selected (a total of 1,568) for inclusion in PCA that had higher than twice the median dispersion, minus the minimum value. The edges of a nearest neighbor graph were generated from computing pairwise distances amongst cells in the first 20 PC dimensions using the approximate nearest neighbors package (ANN) in R (CRAN), setting the k parameter to 25. A first round of clustering with the Louvain modularity-based community detection algorithm³¹ (Modularity Optimizer package implemented in Java) utilized a resolution of 0.01 to generate a total of 10 first-round clusters. Each of these clusters was again subjected to gene selection and PCA. These PCs were evaluated for statistically significant gene expression signals using the Jackstraw method (Chung and Storey, 2014; Macosko et al 2015). At most 15 PCs were used in this second round of clustering by Louvain, with the resolution parameter set at 3. The resulting clusters were compared pairwise for differential expression, as in Macosko et al. 2015, and clusters with fewer than 10 genes differentially expressed by more than 2-fold were merged, producing 202 clusters.

### Electron Microscopy

An 8 month old organoid was immersion-fixed in 2.5% Paraformaldehyde, 2.5% Glutaraldehyde in 0.1M Sodium Cacodylate buffer (EMS #15949) at room temperature for 24 h. The organoid was washed in cold 0.15M Sodium Cacodylate buffer (Caco buffer), embedded in 4% low-melt agarose, and sectioned into 100 micrometer thick slices (Leica Vibratome) in cold Caco buffer (FIG. 8). We selected 3 slices for further processing. The results shown in FIGS. 3 and 8 come from one of those slices.

Preparation for EM was similar to that previously described²⁴. Briefly, sections were washed in cold Caco buffer and stained for 2 h in reduced 2% Osmium Tetroxide solution (with 15 mg/ml Potassium Ferrocyanide) on ice, washed, incubated in 1% Thiocarbohydrazide solution for 30 minutes, washed again, and finally stained with 2% Osmium Tetroxide solution for 1 h (ROTO method). Slices were then dehydrated in a series of ascending Ethanol concentrations, followed by Acetone and Propylene Oxide, infiltrated with mixtures of Epoxy resin (variants of EPON) and Propylene Oxide, and embedded in 100% epoxy resin and cured at 60° C. for two weeks.

Blocks were trimmed and sectioned at 40 nm slice thickness with a Leica EM UC6 Ultramicrotome, and serial sections were collected on carbon-coated Kapton tape using an ATUM (see ²⁴). Strips of tape were mounted on silicon wafers and sections were post-stained with 2% Uranyl Acetate and Lead Aspartate (Leica Ultrastain). Slices were then imaged in a Zeiss Sigma scanning electron microscope. A stack of 136 slice images was acquired using backscatter electron imaging (9 kV, 3 μs dwell time per pixel, 122882 pixels at 4 nm resolution). A roughly aligned region of 40962 pixels was cropped from the images and aligned using ImageJ (“Linear Stack Alignment with SIFT”, using expected transformation: Affine). The resulting aligned image stack was imported into VAST Lite for manual segmentation (freely available on the World Wide Web at subdomain software.rc.fas.harvard.edu/lichtman/vast/). 3D rendering was done in in 3ds Max (Autodesk, Inc). Data analysis was performed in Matlab (The Mathworks, Inc.). For the Wilcoxon rank-sum test we first performed a principal component analysis of the point cloud of each axon and dendrite, and used the vertical components of each largest principal component of all axons vs all dendrites.

### Electrophysiology

Cerebral organoids were transferred from the culture flask to a recording chamber containing artificial cerebrospinal fluid composed of (in mM), NaCl (125.0), D-Glucose (10.0), NaHCO₃ (26.2), KCl (3.5), NaH₂PO₄ (1.3), MgCl₂ (1.0), CaCl₂ (1.2), L-Ascorbic acid (1.0), and Na-Pyruvate (1.0), bubbled with carbogen (95% 02, 5% CO₂). In the recording chamber, the organoid was immobilized by placing it on a small grid of spikes. Prior to recording, the organoids were initially held at 32° C. for 30 minutes, followed by a further 30 minutes acclimatization at 36° C. (the recording temperature). The temperature of the recording chamber was monitored and maintained by a temperature control unit (TC-344C, Warner Instruments), and a custom built heating platform. During acclimatization and electrophysiological recordings, the organoids were maintained in the absence of ambient light. Silicon probes were positioned by a micromanipulator (Scientifica PatchStar) under infrared illumination (850 nm LED) using a digital microscope (Dino-Lite Edge AM4115-FIT). Extracellular neurophysiological signals were recorded using high density silicon probes (Scholvin et al., 2016). Signals were processed and digitized on a headstage proximal to the silicon probe (Intan RHD2000 chip, 30 kS/s per channel, 0.1 Hz high pass filter, 7.6 kHz anti-aliasing filter, 16-bit ADC), then acquired by a NeuroNexus SmartBox system. Wide-band data was stored for analysis. Spike detection was carried out by SpikeDetekt. The initial spike clustering was done by an unsupervised masked EM algorithm (KlustaKwik), followed by a stage of manual curation aided by KlustaViewa software (full procedure is described in Rossant et al., 2016).

Spontaneous neuronal activity was recorded for at least 15 minutes. The 99% confidence bounds for the fano factor were approximated by a gamma distribution³². Mono-synaptic connections were identified by the detection of peaks or troughs, with a short time lag (<5 ms), in the spike-train cross-correlograms (Fujisawa et al., 2008; Stark & Abeles, 2009). The statistical threshold for the identification of connected pairs was set at the 99.9th percentile of the cumulative Poisson distribution for the estimated spike rate the estimated spike rate was calculated by convolving the cross-correlogram (0.5 ms bins) with a Gaussian window (s.d. 10 ms). A cerebral organoid was classified as not active when no spontaneous action potentials were detected during 40 min recording in at least 3 different recording sites.

Stock solutions of 2,3-Dioxo-6-nitro-1,2,3,4-tetrahydrobenzo[f]quinoxaline-7-sulfonamide disodium salt (NBQX disodium salt, Abcam; 100 mM) and Tetrodotoxin citrate (TTX, Abcam; 10 mM) were prepared in dd.H₂0.

Light stimulation was controlled by in-house MATLAB scripts. Briefly, a national instruments board (NI PCIe-6323, X Series DAQ) was used to deliver TTL pulses to an external LED driver (LEDD1B, ThorLabs). For light stimulation, a 530 nm LED (Thorlabs, M530L3) was single band-pass filtered (11 nm bandwidth, Semrock FF01-530/11) and collimated. Using a calibrated meter and photodiode (PM100D and S130C, Thorlabs), the maximum LED power output was set to 300 μW/cm², and the final output controlled by pulse width modulation. During light stimulation, ninety 200 ms pulses of 530 nm light was delivered at 0.2 Hz (the first 30 were 30 μW/cm², the second 30 were at 100 μW/cm², the final 30 were at 300 μW/cm²).

All descriptive statistics and statistical tests were performed in MATLAB R environment (version 8.3, R2014a, The MathWorks, Inc.), using the Statistics Toolbox (version 9.0, R2014a, The MathWorks, Inc.). Mean spike rate data is presented as Tukey style box plots, showing the 1st, 2nd, and 3rd quantile (Q1, Q2, & Q3 respectively; inter-quartile range, IQR=Q3-Q1). Box plot whiskers extend to the most extreme data points between Q1-1.5*IQR and Q3+1.5*IQR 33-35. For illustrative purposes, all data points outside the whiskers are plotted as outliers.

TABLE 1
Primary antibodies employed in this study:

| | Host | | Catalog | |
|---|---|---|---|---|
| Antibody | animal | Company | number | Dilution |
| CTIP2 | Rat | Abcam | AB18465 | 1:100 |
| CUX1 | Mouse | Abcam | AB54583 | 1:350 |
| DCX | Goat | Santa Cruz | SC8066 | 1:300 |
| GFAP | Mouse | Sigma | G3893 | 1:400 |
| GABA | Rabbit | Sigma | A2052 | 1:1000 |
| ISL1 | Rabbit | Abcam | AB109517 | 1:2000 |
| MAP2 | Chicken | Abcam | AB5392 | 1:5000 |
| Nestin | Mouse | Abcam | AB6142 | 1:100 |
| NKX2.1 | Rabbit | DSBS | 74.5A5 | 1:250 |
| OTX2 | Rabbit | Abcam | AB21990 | 1:100 |
| PAX6 | Rabbit | Biolegend | 901301 | 1:400 |
| SYN1 | Mouse | SYSY | 106 001 | 1:100 |
| TH | Rabbit | Millipore | AB152 | 1:5000 |
| TUJ1 | Rabbit | Sigma | T2200 | 1:1000 |
| GBX2 | Goat | Abcam | AB109726 | 1:200 |
| OLIG2 | Rabbit | BBL | 18953 | 1:200 |
| VGLUT1 | Rabbit | SYSY | 135302 | 1:1000 |
| HOMER1 | Rabbit | SYSY | 160003 | 1:700 |
| c-FOS | Rabbit | Abcam | AB134122 | 1:500 |
| RHODOPSIN | Mouse | Abcam | AB5417 | 1:800 |

### REFERENCES

1 Kelava, I. & Lancaster, M. A. Stem Cell Models of Human Brain Development. Cell Stem Cell 18, 736-748, doi:10.1016/j.stem.2016.05.022 (2016).
2 Lancaster, M. A. et al. Cerebral organoids model human brain development and microcephaly. Nature 501, 373-379, doi:nature12517 [pii] 10.1038/nature12517 [doi] (2013).
3 Cugola, F. R. et al. The Brazilian Zika virus strain causes birth defects in experimental models. Nature 534, 267-271, doi:10.1038/nature18296 (2016).
4 Dang, J. et al. Zika Virus Depletes Neural Progenitors in Human Cerebral Organoids through Activation of the Innate Immune Receptor TLR3. Cell Stem Cell, doi:10.1016/j.stem.2016.04.014 (2016).
5 Garcez, P. P. et al. Zika virus impairs growth in human neurospheres and brain organoids. Science, doi:10.1126/science.aaf6116 (2016).
6 Nowakowski, T. J. et al. Expression Analysis Highlights AXL as a Candidate Zika Virus Entry Receptor in Neural Stem Cells. Cell Stem Cell, doi:10.1016/j.stem.2016.03.012 (2016).
7 Qian, X. et al. Brain-Region-Specific Organoids Using Mini-bioreactors for Modeling ZIKV Exposure. Cell 165, 1238-1254, doi:10.1016/j.cell.2016.04.032 (2016).
8 Mariani, J. et al. FOXG1-Dependent Dysregulation of GABA/Glutamate Neuron Differentiation in Autism Spectrum Disorders. Cell 162, 375-390, doi:10.1016/j.ce11.2015.06.034 (2015).
9 Macosko, E. Z. et al. Highly Parallel Genome-wide Expression Profiling of Individual Cells Using Nanoliter Droplets. Cell 161, 1202-1214, doi:10.1016/j.ce11.2015.05.002 (2015).
10 Satija, R., Farrell, J. A., Gennert, D., Schier, A. F. & Regev, A. Spatial reconstruction of single-cell gene expression data. Nat Biotechnol 33, 495-502, doi:10.1038/nbt.3192 (2015).
11 Amir, E. D. et al. viSNE enables visualization of high dimensional single-cell data and reveals phenotypic heterogeneity of leukemia. Nat Biotechnol 31, 545-552, doi:10.1038/nbt.2594 (2013).
12 van der Maaten, L. a. H., Geoffrey. Visualizing Data using t-SNE Journal of Machine Learning Research 9, 2579-2605 (2008).
13 Arlotta, P. et al. Neuronal subtype-specific genes that control corticospinal motor neuron development in vivo. Neuron 45, 207-221, doi:S0896627304008530 [pii] 10.1016/j.neuron.2004.12.036 [doi] (2005).
14 Zhang, Y. et al. Purification and Characterization of Progenitor and Mature Human Astrocytes Reveals Transcriptional and Functional Differences with Mouse. Neuron 89, 37-53, doi:10.1016/j.neuron.2015.11.013 (2016).
15. Marques, S. et al. Oligodendrocyte heterogeneity in the mouse juvenile and adult central nervous system. Science 352, 1326-1329, doi:10.1126/science.aaf6463 (2016).
16 Zhang, Y. et al. An RNA-sequencing transcriptome and splicing database of glia, neurons, and vascular cells of the cerebral cortex. J Neurosci 34, 11929-11947, doi:10.1523/jneurosci.1860-14.2014 (2014).
17 Pollen, A. A. et al. Molecular identity of human outer radial glia during cortical development. Cell 163, 55-67, doi:10.1016/j.cell.2015.09.004 (2015).
18 Furukawa, T., Morrow, E. M. & Cepko, C. L. Crx, a novel otx-like homeobox gene, shows photoreceptor-specific expression and regulates photoreceptor differentiation. Cell 91, 531-541 (1997).
19 Korf, H. W., White, B. H., Schaad, N. C. & Klein, D. C. Recoverin in pineal organs and retinae of various vertebrate species including man. Brain Res 595, 57-66 (1992).
20 Siegert, S. et al. Transcriptional code and disease map for adult retinal cell types. Nat Neurosci 15, 487-495, s481-482, doi:10.1038/nn.3032 (2012).
21 Strunnikova, N. V. et al. Transcriptome analysis and molecular signature of human retinal pigment epithelium. Hum Mol Genet 19, 2468-2486, doi:10.1093/hmg/ddq129 (2010).
22 Dennis, G., Jr. et al. DAVID: Database for Annotation, Visualization, and Integrated Discovery. Genome Biol 4, P3 (2003).
23 Clarke, L. E. & Barres, B. A. Emerging roles of astrocytes in neural circuit development. Nat Rev Neurosci 14, 311-321, doi:10.1038/nrn3484 (2013).
24 Kasthuri, N. et al. Saturated Reconstruction of a Volume of Neocortex. Cell 162, 648-661, doi:10.1016/j.ce11.2015.06.054 (2015).
25 Schizophrenia Working Group of the Psychiatric Genomics Consortium. Biological insights from 108 schizophrenia-associated genetic loci. Nature 511, 421-427, doi:10.1038/nature13595 (2014).
26 Sanders, S. J. et al. Insights into Autism Spectrum Disorder Genomic Architecture and Biology from 71 Risk Loci. Neuron 87, 1215-1233, doi:10.1016/j.neuron.2015.09.016 (2015).
27 Boulting, G. L. et al. A functionally characterized test set of human induced pluripotent stem cells. Nat Biotechnol 29, 279-286, doi:10.1038/nbt.1783 (2011).
28 Chen, A. E. et al. Optimal timing of inner cell mass isolation increases the efficiency of human embryonic stem cell derivation and allows generation of sibling cell lines. Cell Stem Cell 4, 103-106, doi:10.1016/j.stem.2008.12.001 (2009).
29 Lancaster, M. A. & Knoblich, J. A. Generation of cerebral organoids from human pluripotent stem cells. Nat Protoc 9, 2329-2340, doi:10.1038/nprot.2014.158 (2014).
30 Quadrato, G. et al. Nuclear factor of activated T cells (NFATc4) is required for BDNF-dependent survival of adult-born neurons and spatial memory formation in the hippocampus. Proc Natl Acad Sci USA 109, E1499-1508, doi:10.1073/pnas 0.1202068109 (2012).
31 Waltman, L. A., Eckel-Passow, J. E., Sharma, R. G. & Van Dyke, D. L. Advanced maternal age in polyploidy with concurrent aneuploidy. American journal of medical genetics. Part A 161A, 1200-1202, doi:10.1002/ajmg.a.35870 (2013).
32 Eden, U. T. & Kramer, M. A. Drawing inferences from Fano factor calculations. J Neurosci Methods 190, 149-152, doi:10.1016/j.jneumeth.2010.04.012 (2010).
33 McGill, R., Tukey, J. W. & Larsen, W. A. Variations of box plots. The American Statistician 32, 12-16 (1978).
34 Krzywinski, M. & Altman, N. Visualizing samples with box plots. Nat Methods 11, 119-120 (2014).
35 Streit, M. & Gehlenborg, N. Bar charts and box plots. Nat Methods 11, 117 (2014).
36 Camp, J. G. et al. Human cerebral organoids recapitulate gene expression programs of fetal neocortex development. Proc Natl Acad Sci USA 112, 15672-15677, doi:10.1073/pnas.1520760112 (2015).
37 Cahoy, J. D. et al. A transcriptome database for astrocytes, neurons, and oligodendrocytes: a new resource for understanding brain development and function. The Journal of neuroscience: the official journal of the Society for Neuroscience 28, 264-278, doi:10.1523/JNEUROSCI.4178-07.2008 (2008).
38 Xia, N. et al. Transcriptional comparison of human induced and primary midbrain dopaminergic neurons. Sci Rep 6, 20270, doi:10.1038/srep20270 (2016).
39 Molyneaux, B. J. et al. Novel subtype-specific genes identify distinct subpopulations of callosal projection neurons. J Neurosci 29, 12343-12354, doi:29/39/12343 [pii]10.1523/JNEUROSCI.6108-08.2009 [doi] (2009).
40 Darmanis, S. et al. A survey of human brain transcriptome diversity at the single cell level. Proc Natl Acad Sci USA 112, 7285-7290, doi:10.1073/pnas.1507125112 (2015).
41 Lake, B. B. et al. Neuronal subtypes and diversity revealed by single-nucleus RNA sequencing of the human brain. Science 352, 1586-1590, doi:10.1126/science.aaf1204 (2016).

1.-41. (canceled)

42. A three dimensional neural tissue composition comprising a cerebral organoid exhibiting discrete brain regions comprising sensory cells, wherein the sensory cells comprise one or more photoreceptors capable of detecting a light stimulus.

43. The three dimensional neural tissue composition according to claim 42, wherein the photoreceptors are capable of responding to a detected light stimulus.

44. The three dimensional neural tissue composition according to claim 42, further comprising a neural circuit.

45. The three dimensional neural tissue composition according to claim 44, wherein the sensory cells form a neural network with one or more additional cells within the organoid.

46. The three dimensional neural tissue composition according to claim 45, wherein the neural network includes a functional connection between the neural circuit and the sensory cell.

47. The three dimensional neural tissue composition according to claim 45, wherein the neural network is capable of exhibiting a response to light.

48. The three dimensional neural tissue composition according to claim 42, wherein the sensory cells comprise at least about 1%, at least about 5%, at least about 10% or at least about 20% of the population of cells of the cerebral organoid.

49. The three dimensional neural tissue composition according to claim 42, wherein the cerebral organoid is a mature cerebral organoid exhibiting dendritic spine-like structures.

50. The three dimensional neural tissue composition according to claim 42, comprising spontaneously-active neurons and neuronal networks.

51. The three dimensional neural tissue composition according to claim 42, wherein the sensory cells express one or more markers indicative of retinal cells.

52. The three dimensional neural tissue composition according to claim 42, wherein the sensory cells express a marker selected from the group consisting of CRX, RCVN and NRL.

53. An in vitro method of producing a three dimensional neural tissue composition comprising:
forming embryoid bodies from cells;
applying a medium comprising hESC medium and neural induction medium to the formed embryoid bodies;
generating neuroectodermal tissue from the embryoid bodies;
transferring the neuroectodermal tissue to a protein mixture and maintaining in a cerebral organoid differentiation medium for 3 to 5 days to form neural tissue;
transferring the neural tissue to a tissue culture vessel and maintaining in the cerebral organoid differentiation medium for 28 to 32 days; and
replacing the cerebral organoid differentiation medium with a cerebral organoid differentiation medium supplemented with neurotrophin BDNF and maintaining neural tissue in the supplemented organoid differentiation medium for a time sufficient to produce a three dimensional neural tissue composition comprising a cerebral organoid exhibiting discrete brain regions comprising sensory cells having photoreceptors, wherein said one or more photoreceptors are capable of detecting a light stimulus.

54. A method according to claim 53, wherein the cells are human cells.

55. A method according to claim 53, wherein the cells are human stem cells.

56. A brain organoid-machine interface comprising:
a multi-probe electrode array configured to collect electrophysiological signals from neural tissue;
a first processor operably linked to the multi-probe electrode array;
a second processor operably linked to a stimulus-generating device, wherein the stimuls-generating device is a light-emitting diode (LED); and
machine executable instructions configured to decode circuit response and instruct feedback stimulation to the sensory generating device.

57. The brain organoid-machine interface according to claim 56, wherein the first processor is configured to collect and store the electrophysiological signals.

58. The brain organoid-machine interface according to claim 56, wherein the stimulus-generating device generates a visual stimulus.

59. The brain organoid-machine interface according to claim 56, wherein the brain organoid-machine interface measures the neural tissue for spontaneous activity or network activity in response to the stimulus.

60. The brain organoid-machine interface according to claim 56, wherein the brain organoid-machine interface monitors the neural tissue for dysregulation of spontaneous activity in response to the stimulus.

61. The brain organoid-machine interface according to claim 56, further comprising neural tissue operably linked to the multi-probe electrode array and comprising a cerebral organoid exhibiting discrete brain regions comprising one or more photoreceptors and cells, wherein said one or more photoreceptors are capable of detecting a visual stimulus.
